(19) United States
(12) Patent Application Publication
(10) Pub. No.: US 2011/0112193 A1
(43) Pub. Date: May 12, 2011

(54) BIS-ARYL COMPOUNDS FOR USE AS MEDICAMENTS

(76) Inventors: Peter Nilsson, Uppsala (SE); Benjamin Pelcman, Uppsala (SE); Martins Katkevics, Riga (LV)

(21) Appl. No.: 12/992,074

(22) PCT Filed: May 5, 2009
(86) PCT No.: PCT/GB2009/001227

§ 371 Date: Jan. 24, 2011

Related U.S. Application Data
(60) Provisional application No. 61/071,715, filed on May 14, 2008.

(51) Int. Cl. A61K 31/255; C07C 311/35; C07C 229/64; A61K 31/196; C07C 303/36; C07C 227/16; A61P 11/00; A61P 29/00; A61P 11/06; A61P 37/08; A61P 17/00; A61P 27/02; A61P 25/00; A61P 13/00; A61P 5/00; A61P 31/04; A61P 31/12; A61P 31/10; A61P 7/06; A61P 35/00

(52) U.S. Cl. 514/562; 562/430; 562/452; 514/567

(57) ABSTRACT

There is provided compounds of formula (I), wherein ring A, D₁, D₂ₐ, D_{2b}, D₃, L¹, Y¹, L³ and Y³ have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

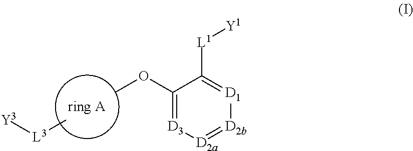

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. They may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRAs) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRAs, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRAs have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

Various polymers have been disclosed in journal articles Polymer 47 (2006), 6606-6621 by S. B. Lee at al, Journal of Polymer Science, Part A: Polymer Chemistry (1994), 32(2), 355-62 by H. R. Kricheldorf at al, as well as in Japanese patent application JP 2006-176495. However, there is no mention in these documents that the compounds disclosed therein may be useful as medicaments.

Journal article Justus Liebigs Annalen der Chemie (1934), 513, 156-79 by H. Liebermann et al discloses the synthesis of various compounds including some biaryls. German patent application discloses the synthesis of various phosphonium phenolates that may be useful as transesterification catalysts, and which may be prepared from various phenols, including biaryl phenols. Further, international patent application WO 02/062870 discloses various polycarbonates that potentially have good hue and melt properties. However, there is no mention in any of these documents that the compounds disclosed therein may be useful as medicaments.

International patent application WO 2007/113337 discloses a fluorescence based test system, which is employed to measure the formation of the HIV gp41 six-helix bundle. Various biaryl compounds in which a carboxylic acid group is meta relative to the linking point of the biaryl core were the subject of such a test. Further international patent application WO 03/075907 discloses various biaryl compounds that may be useful in inhibiting the entry process of the HIV virus into a mammalian host cell. However, there is no mention in either of these documents of biaryl compounds in which there is a carboxylic acid (or variant thereof) ortho to the linkage point of the biaryl ring system. Further, there is no mention that the compounds disclosed therein may be useful in the treatment of inflammation.

International patent application WO 2005/075410 discloses various compounds for use as medicaments. However, this document does not disclose biaryl ring systems, in which each aromatic ring is further substituted (directly or via a linker group) with another aromatic group.

US patent application US 2005/0014169 and international patent application WO 2004/076640 both disclose various biaryl compounds that may act as nuclease inhibitors, with the latter document further stating that the compounds disclosed therein may be useful in the treatment of cancer. However, there is no mention in either document of biaryl compounds in which there is a carboxylic acid (or variant thereof) ortho to the linkage point of the biaryl core, nor that the compounds disclosed therein may be useful in the treatment of inflammation.

International patent application WO 2006/125593 and European patent application EP 1 113 000 both disclose compounds that may have potential use in the treatment of inflammation. However, the former document predominantly relates to biaryl ring systems that are not further substituted with aromatic groups, and the latter mainly relates to biaryl compounds containing a cycloalkylamido moiety, but not a carboxylic acid group, or isostere thereof.

International patent applications WO 2007/113254, WO 2005/053609, WO 01/066098, WO 2006/104957, WO 2006/055625, WO 2005/042520 and WO 01/023347 as well as US patent applications U.S. Pat. No. 6,251,917, US 2004/0229891, US 2004/0082641, US 2005/0277640 and US 2007/0066660 all disclose various biaryl compounds. However, none of these documents mention that the compounds disclosed therein may be useful as inhibitors of LTC₄ synthase, and therefore of use in the treatment of inflammation.

US patent application US 2004/0209882 discloses various methods and compositions of triazine compounds, which may be useful in treating pathophysiological conditions. However, there is no specific disclosure in this document of two aromatic groups linked with an oxygen atom, each of which aromatic groups are further substituted with an aromatic group.

Japanese patent application JP 3056431 discloses compounds containing two phenyl groups linked by way of a carbon, oxygen or sulfur atom, which may be useful in treating inflammatory diseases (e.g. arthritis). However, there is no specific disclosure in this document of two aromatic groups linked with an oxygen or sulfur atom, each of which aromatic groups are further substituted with an aromatic group.

International patent application WO 2009/030887 discloses various compounds for use as LTC₄ inhibitors. However, there is no mention in that document of biaryl compounds that are linked via a heteroatom. Unpublished international patent application PCT/GB2009/000966 discloses various compounds in which the two aromatic groups may be linked together with an oxygen atom. However, this document mainly relates to compounds in which one of those aromatic rings is substituted at the meta position (with respect to the linker group) with a carboxylic acid group (or isostere thereof).

### DISCLOSURE OF THE INVENTION

According to the invention, there is provided a compound of formula I,

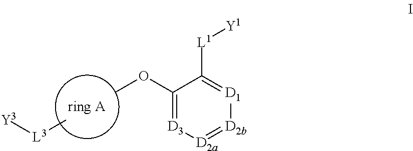

wherein

either one of D₂ₐ and D_{2b} represents D₂, and the other represents —C(-L²-Y²)═;

each of D₁, D₂ and D₃ respectively represent —C(R^{1a})═, C(R^{1b})═ and —C(R^{1c})═, or,

each of D₁, D₂ and D₃ may alternatively and independently represent —N═;

ring A represents:

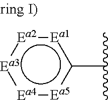

each of E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, or, each of E^{a1}, E^{a2}, E^{a3}, E^{a4} and E^{a5} may alternatively and independently represent —N═;

R^{2a} and R^{2e} independently represent hydrogen, -L^{1a}-Y^{1a} or a substituent selected from X¹;

one of R^{2b}, R^{2c} and R^{2d} represents the requisite -L³-Y³ group, and the others independently represent hydrogen, -L^{1a}-Y^{1a} or a substituent selected from X¹;

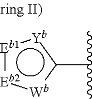

E^{b1} and E^{b2} respectively represent —C(R^{3a})═ and —C(R^{3b})═;

Y^{b} represents —C(R^{3c})═ or —N═;

W^{b} represents —N(R^{3d})—, —O— or —S—;

one of R^{3a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or

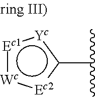

E^{c1} and E^{c2} each respectively represent —C(R^{4a})═ and —C(R^{4b})═;

Y^{c} represents —C(R^{4c})═ or —N═;

W^{c} represents —N(R^{4d})—, —O— or —S—;

one of R^{4a}, R^{4b} and, if present, R^{4c} and R^{4d} represents the requisite -L³-Y³ group, and the remaining R^{4a}, R^{4b} and (if present) R^{4c} substituents represent hydrogen, -L^{1a}-Y^{1a} or a substituent selected from X³, and the remaining R^{4d} substituent (if present) represents hydrogen or a substituent selected from R^{z2};

R^{z1} and R^{z2} independently represent a group selected from Z^{1a};

R^{1a}, R^{1b} and R^{1c} independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R⁶)S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;

X¹, X² and X³ independently represent a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;

Z^{1a} and Z^{2a} independently represent, on each occasion when used herein, —R^{5a}, —C(O)R^{5b}, —C(O)OR^{5c}, —C(O)N(R^{6a})R^{7a}, —S(O)ₘR^{5j} or —S(O)₂N(R^{6h})R^{7h};

R^{5b} to R^{5h}, R^{5j}, R^{5k}, R⁵ⁿ, R^{6a} to R⁶ⁱ, R^{7a}, R^{7b}, R^{7d} and R^{7f} to R⁷ⁱ independently represent, on each occasion when used herein, H or R^{5a}; or

any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O, —OR^{5h} and R^{5a};

R⁵ⁱ, R^{5m} and R^{5p} independently represent R^{5a};

R^{5a} represents, on each occasion when used herein, C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and —OS(O)₂N(R^{8g})R^{8h};

n represents 0, 1 or 2;

R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and —S(O)₂-M¹;

R^{8c}, R^{8f} and R^{8h} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14b} and —S(O)₂-M²; or

R^{8b} and R^{8c}, R^{8e} and R^{8f} or R^{8g} and R^{8h} may be linked together to form, along with the atom(s) to which they are attached, a 3- to 6-membered ring, which ring optionally contains a further heteroatom (such as nitrogen or oxygen) in addition to the nitrogen atom to which these substituents are necessarily attached, and which ring is optionally substituted by one or more substituents selected from F, Cl, ═O or C₁₋₃ alkyl optionally substituted by one or more substituents selected from ═O and fluoro;

M¹ and M² independently represent —CH₃, —CH₂CH₃, —CF₃ or —N(R^{15a})R^{15b};

R^{11a} and R^{13a} independently represent H, —CH₃, —CH₂CH₃, —CF₃ or —CHF₂;

R^{12a}, R^{12b}, R^{14a}, R^{14b}, R^{15a} and R^{15b} independently represent H, —CH₃ or —CH₂CH₃,

Y¹ and Y^{1a} independently represent, on each occasion when used herein, —N(H)SO₂R^{9a}, —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f}), —P(O)(N(R^{10g})R^{9g})₂, —B(OR^{9h})₂, —C(CF₃)₂OH, —S(O)₂N(R¹⁰ⁱ)R⁹ⁱ or any one of the following groups:

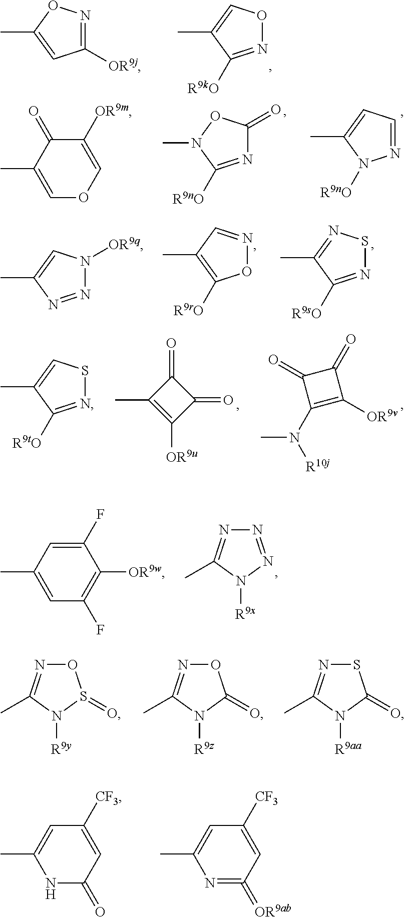

R^{9a} represents on each occasion when used herein, C₁₋₈ alkyl, a heterocycloalkyl group, an aryl group or a heteroaryl group which are optionally substituted by one or more substituents selected from G¹ and/or Z¹;

R^{9b} to R^{9z}, R^{9aa}, R^{9ab}, R^{10f}, R^{10g}, R¹⁰ⁱ and R^{10j} independently represent, on each occasion when used herein, C₁₋₈ alkyl or a heterocycloalkyl group, both of which are optionally substituted by one or more substituents selected from G¹ and/or Z¹; or

R^{9b} to R^{9z}, R^{9aa}, R^{9ab}, R^{10f}, R^{10g}, R¹⁰ⁱ and R^{10j} independently represent, on each occasion when used herein, hydrogen; or

any pair of R^{9f} and R^{10f}, R^{9g} and R^{10g}, and R⁹ⁱ and R¹⁰ⁱ, may be linked together to form, along with the atom(s) to which they are attached, a 3- to 6-membered ring, which ring optionally contains a further heteroatom (such as nitrogen or oxygen), in addition to the nitrogen atom to which these substituents are necessarily attached, and which ring is optionally substituted by one or more substituents selected from F, Cl, ═O, —OR^{5h} and R^{5a};

Y² and Y³ independently represent an aryl group or a heteroaryl group, both of which groups are optionally substituted by one or more substituents selected from A;

A represents, on each occasion when used herein:

I) an aryl group or a heteroaryl group, both of which are optionally substituted by one or more substituents selected from B;

II) C₁₋₈ alkyl or a heterocycloalkyl group, both of which are optionally substituted by one or more substituents selected from G¹ and/or Z¹; or

III) a G¹ group;

G¹ represents, on each occasion when used herein, halo, cyano, —N₃, —NO₂, —ONO₂ or -A¹-R^{16a};

wherein A¹ represents a single bond or a spacer group selected from —C(O)A²-, —S—, —S(O)ᵣA³-, —N(R^{17a})A⁴- or —OA⁵-, in which:

A² represents a single bond, —O—, —N(R^{17b})— or —C(O)—;

A³ represents a single bond, —O— or —N(R^{17c})—;

A⁴ and A⁵ independently represent a single bond, —C(O)—, —C(O)N(R^{17d})—,

—C(O)O—, —S(O)ᵣ— or —S(O)ᵣN(R^{17e})—;

Z¹ represents, on each occasion when used herein, ═O, ═S, ═NOR^{16b}, ═NS(O)₂N(R^{17f})R^{16c}, ═NCN or ═C(H)NO₂;

B represents, on each occasion when used herein:

I) an aryl group or a heteroaryl group, both of which are optionally substituted by one or more substituents selected from G²;

II) C₁₋₈ alkyl or a heterocycloalkyl group, both of which are optionally substituted by one or more substituents selected from G² and/or Z²; or

III) a G² group;

G² represents, on each occasion when used herein, halo, cyano, —N₃, —NO₂, —ONO₂ or -A⁶-R^{18a};

wherein A⁶ represents a single bond or a spacer group selected from —C(O)A⁷-, —S—, —S(O)ᵣA⁸-, —N(R^{19a})A⁹- or —OA¹⁰-, in which:

A⁷ represents a single bond, —O—, —N(R^{19b})— or —C(O)—;

A⁸ represents a single bond, —O— or —N(R^{19e})—;

A⁹ and A¹⁹ independently represent a single bond, —C(O)—, —C(O)N(R^{19d})—, —C(O)O—, —S(O)ᵣ— or —S(O)ᵣN(R^{19e})—;

Z² represents, on each occasion when used herein, ═O, ═S, ═NOR^{18b}, ═NS(O)₂N(R^{19f})R^{18c}, ═NCN or ═C(H)NO₂;

R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:

i) hydrogen;

ii) an aryl group or a heteroaryl group, both of which are optionally substituted by one or more substituents selected from G³;

iii) C₁₋₈ alkyl or a heterocycloalkyl group, both of which are optionally substituted by one or more substituents selected from G³ and/or Z³; or any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;

G³ represents, on each occasion when used herein, halo, cyano, —N₃, —NO₂, —ONO₂ or -A¹¹-R^{29a};

wherein A¹¹ represents a single bond or a spacer group selected from —C(O)A¹²-, —S—, —S(O)ᵣA¹³-, —N(R^{21a})A¹⁴- or —OA¹⁵-, in which:

A¹² represents a single bond, —O—, —N(R^{21b})— or —C(O)—;

A¹³ represents a single bond, —O— or —N(R^{21c})—;

A¹⁴ and A¹⁵ independently represent a single bond, —C(O)—, —C(O)N(R^{21d})—, —C(O)O—, —S(O)ᵣ— or —S(O)ᵣN(R^{21e})—;

Z³ represents, on each occasion when used herein, ═O, ═S, ═NOR^{20b}, ═NS(O)₂N(R^{21f})R^{20c}, ═NCN or ═C(H)NO₂;

each r independently represents, on each occasion when used herein, 1 or 2;

R^{20a}, R^{20b}, R^{20c}, R^{21a}, R^{21b}, R^{21c}, R^{21d}, R^{21e} and R^{21f} are independently selected from:

i) hydrogen;

ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and

iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or

any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;

L¹ and L^{1a} independently represent a single bond or C₁₋₆ alkylene in which any one of the carbon atoms may be replaced by Q;

Q represents —C(R^{y1})(R^{y2})—, —C(O)— or —O—;

R^{y1} and R^{y2} independently represent H, F or X⁴; or

R^{y1} and R^{y2} may be linked together to form a 3- to 6-membered ring, which ring optionally contains a heteroatom, and which ring is optionally substituted by one or more substituents selected from F, Cl, ═O and X⁵;

L² and L³ independently represent a single bond or a spacer group selected from —(CH₂)ₚ—C(R^{y3})(R^{y4})—(CH₂)_{q}-A¹⁶-, —(CH₂)ₚ—C(O)A¹⁷-, —(CH₂)ₚ—S—, —(CH₂)ₚ—SC(R^{y3})(R^{y4})—, —(CH₂)ₚ—S(O)A²¹-, —(CH₂)ₚ—S(O)₂A¹⁸-, —(CH₂)ₚ—N(R^{w})A¹⁹- or —(CH₂)ₚ—OA²⁰-, in which:

A¹⁶ represents a single bond, —O—, —N(R^{w})—, —C(O)—, or —S(O)ₘ—;

A¹⁷, A¹⁸ and A²¹ independently represent a single bond, —C(R^{y3})(R^{y4})—, —O—, —N(R^{w})— or —N(R^{w})SO₂—;

A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;

p and q independently represent, on each occasion when used herein, 0, 1 or 2;

m represents, on each occasion when used herein, 0, 1 or 2;

R^{y3} and R^{y4} independently represent, on each occasion when used herein, H, F or X⁶; or

R^{y3} and R^{y4} may be linked together to form a 3- to 6-membered ring, which ring optionally contains a heteroatom, and which ring is optionally substituted by one or more substituents selected from F, Cl, ═O and X⁷;

R^{w} represents, on each occasion when used herein, H or X⁸;

X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, C₁₋₄ alkyl (optionally substituted by one or more substituents selected from fluoro, chloro and ═O), —N(R^{26a})R^{26b}, —OR^{26c} and —C(O)R^{26d});

R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b}, R^{26c} and R^{26d} are independently selected from hydrogen and C₁₋₄ alkyl, which latter group is optionally substituted by one or more substituents selected from fluoro, chloro and/or ═O,

or a pharmaceutically-acceptable salt thereof, provided that when L¹ represents a direct bond; Y¹ represents —C(O)OH; ring A represents ring I):

(I) D₁, D₂ₐ and D₃ all represent —C(—COOH)═; D_{2b} represents —C(-L²-Y²)═; E^{a1}, E^{a2}, E^{a4} and E^{a5} all represent —C(H)═; E^{a3} represents —C(R^{2c})═; R^{2c} represents the requisite -L³-Y³ group; L² represents —O—; Y² represents phenyl substituted in the 4-position by A; A represents phenyl substituted in the 4-position by G²; L³ represents a direct bond; Y³ represents phenyl substituted in the 4-position by A; A represents G¹, then G¹ and G² do not both represent dodecyloxy, decyloxy, octyloxy or hexyloxy;

(II) D₁ and D₃ both represent —C(H)═; D₂ₐ represents —C(—COOH)═; R^{1b} represents —COOH; D_{2b} represents —C(-L²-Y²)═; E^{a1}, E^{a4} and E^{a5} all represent —C(H)═; L² represents —O—:
(a) Y² represents phenyl substituted in the 3-position by —O—CH₂-phenyl and in the 4-position by —NO₂; E^{a3} represents —C(NO₂)═; E^{a2} represents —C(R^{2b})═; R^{2b} represents the requisite -L³-Y³ group; L³ represents —OCH₂—, then Y³ does not represent unsubstituted phenyl;
(b) Y² represents phenyl substituted in the 4-position by —S(O)₂-phenyl; E^{a2} represents —C(H)═; Ea³ represents —C(R^{2c})═; R^{2c} represents the requisite -L³-Y³ group; L³ represents —S(O)₂—, then Y³ does not represent unsubstituted phenyl;

(III) D₁ and D₃ both represent —C(OH)═; D₂ₐ represents —C(—COOH)═; D_{2b} represents —C(-L²-Y²)═; L² represents —O—; E^{a1}, E^{a2}, E^{a4} and E^{a5} all represent —C(H)′; E^{a3} represents —C(R^{2c})═; R^{2c} represents the requisite -L³-Y³ group; L³ represents a single bond, then:
(a) Y³ does not represent unsubstituted phenyl when Y² represents (4-phenyl)phenyl;
(b) Y³ does not represent 4-hydroxyphenyl when Y² represents [(4-hydroxy)phenyl]phenyl;

(IV) D₁, D₂ₐ and D₃ all represent —C(H)═; D_{2b} represents —C(-L²-Y²)═; E^{a2}, E^{a4} and E^{a5} all represent —C(H)═; E^{a3} represents —C(R^{2c})═; R^{2c} represents the requisite -L³-Y³ group; L² and L³ both represents —C(CH₃)₂—; then Y² and Y³ do not both represent 4-hydroxyphenyl when:
(a) E^{a1} represents hydrogen;
(b) E^{a1} represents —C(-L^{1a}-Y¹)═ and -L^{1a}-Y^{1a} represents —COOH,

which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range), defined herein may be straight-chain or, when there is a sufficient number (i.e. a minimum of two or three, as appropriate) of carbon atoms, be branched-chain, and/or cyclic (so forming, in the case of alkyl, a C_{3-q} cycloalkyl group or, in the case of alkylene, a C_{3-q} cycloalkylene group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, in the case of alkyl, a C_{2-q} alkenyl or a C_{2-q} alkynyl group or, in the case of alkylene, a C_{2-q} alkenylene or a C_{2-q} alkynylene group). In the case of alkylene groups, it is preferred that they are acyclic and/or straight-chain, but may be saturated or unsaturated.

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothiadiazolyl (including 2,1,3-benzothiadiazolyl), benzothienyl, carbazolyl, chromanyl, cinnolinyl, furanyl, imidazolyl, imidazopyridyl (including imidazo[4,5-b]pyridyl, imidazo[5,4-b]pyridyl and imidazo[1,2-a]pyridyl), indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,3,4-thiadiazolyl), thiazolyl, oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl), thiochromanyl, thienyl, triazolyl (including 1,2,3-triazolyl and 1,2,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic, they are preferably linked to the rest of the molecule via an aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which two X¹ groups are present, which both represent R^{5a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when there are two X¹ substituents present, which represent —R^{5a} and —C(O)R^{5b} in which R^{5b} represents R^{5a}, then the identities of the two R^{5a} groups are not to be regarded as being interdependent. Likewise, when Y² or Y³ represent e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{5a} to R^{5h}” is employed herein, this will be understood by the skilled person to mean R^{5a}, R^{5b}, R^{5c}, R^{5d}, R^{5e}, R^{5f}, R^{5g} and R^{5h} inclusively.

For the avoidance of doubt, when the term “an R⁵ group” is referred to herein, we mean any one of R^{5a} to R^{5k}, R^{5m}, R⁵ⁿ or R^{5p}.

For the avoidance of doubt, where it is stated herein that “any pair of R^{16a} to R^{16c} and R^{17a} to R^{17f} . . . may . . . be linked together”, we mean that any one of R^{16a}, R^{16b} or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—) or R^{16c} and R^{17f} may be linked together with the nitrogen atom to which they are necessarily attached to form a ring as hereinbefore defined.

For the avoidance of doubt, the compounds of the invention relate to either of the following compounds of formula I,

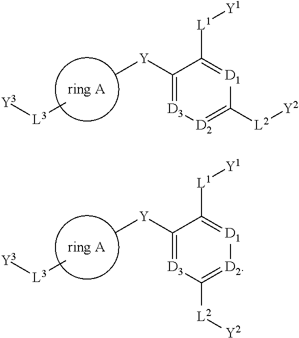

The skilled person will appreciate that, given that there is an essential ‘-L³-Y³’ group present in the compound of formula I, then when, for example, ring A represents ring I), then at least one of —C(R^{2b})═, —C(R^{2c})═ and —C(R^{2d})═ must be present, in which the any one of the relevant R^{2b}, R^{2c} and R^{2d} groups represents the essential -L³-Y³ group.

When L¹ or L^{1a} represents C₁₋₆ alkylene in which any one of the carbon atoms is replaced with Q, it is preferred that the C₁₋₆ alkylene group is interrupted by Q. That it, it may represent —C_{q1}(alkylene)-Q-C_{q2}(alkylene), in which the sum of q1 and q2 equals 6, provided that neither q1 nor q2 represents 0. Preferably, the sum of q1 and q2 equals 3.

Compounds of the invention that may be mentioned include those in which:

each r independently represents, on each occasion when used herein, 2;

L² and L³ independently represent a single bond or a spacer group selected from —(CH₂)ₚ—C(R^{y3})(R^{y4})—(CH₂)_{q}-A¹⁶-, —(CH₂)ₚ—C(O)A¹⁷-, —(CH₂)ₚ—S—, —(CH₂)ₚ—SC(R^{y3})( R^{y4})—, —(CH₂)ₚ—S(O)₂A¹⁸-, —(CH₂)ₚ—N(R^{w})A¹⁹- or —(CH₂)ₚ—OA²⁰-.

Compounds of the invention that may be mentioned include those in which for example when:

when R^{5a} or R^{8a} to R^{8h} represents optionally substituted C₁₋₆ alkyl, then preferably they are not substituted with both ═O and —OR^{8a}, ═O and —OR^{11a}, or ═O and —OR^{13a} (as appropriate) at the terminal positions of the alkyl group (so forming, for example a —C(O)OR^{8a}, —C(O)OR^{11a} or —C(O)OR^{13a} group);

when R^{5a} or R^{8a} to R^{8h} represents optionally substituted C₁₋₆ alkyl, then preferably they are not substituted with both ═O and —N(R^{8b})R^{8c}, ═O and —N(R^{12a})R^{12b}, or ═O and —N(R^{14a})R^{14b} (as appropriate) at the terminal positions of the alkyl group (so forming, for example a —C(O)N(R^{8b})R^{8c}, —C(O)N(R^{12a})R^{12b} or —C(O)N(R^{14a})R^{14b} group);

when alkyl groups defined herein are substituted with one or more halo atoms, then the halo atoms are preferably fluoro.

Compounds of the invention that may be mentioned include those in which, for example, when D₁, D₂ and D₃ respectively represent —C(R^{1a})═, —C(R^{1b})═ and —C(R^{1c})═; ring A represents ring (I) and E^{a1}, E^{a2}, E^{a3}, E^{a4} and E^{a5} respectively represent —C(R^{2a})═, —C(R^{2b})═, —C(R^{2d})═ and —C(R^{2e})═, then:

when e.g. Y² and Y³ both represent a heteroaryl (e.g. a 4- to 10-membered heteroaryl) group, then L¹ and, if present, L^{1a}, independently represent a single bond, C₁₋₆ alkylene in which any one of the carbon atoms is interrupted by Q, or C₁₋₆ alkylene in which any one of the carbon atoms is replaced with —C(O)— or —C(R^{y1})(R^{y2})—;

when e.g. Y² and Y³ both represent a heteroaryl group, then L² and L³ do not both represent single bonds.

Further compounds of the invention that may be mentioned include those in which, for example, when D₂ₐ represents D₂, and D₁ and D₂ respectively represent —C(R^{1a})═ and —C(R^{1b})═, then:

R^{1a} and/or R^{1b} do not represent —C(O)OR^{5c}, —N(R^{5k})S(O)₂R^{5m}, —C(H)(CF₃)OH, —C(O)CF₃, —C(OH)₂CF₃, —C(CF₃)₂OH or —S(O)₂N(R^{6h})R^{7h} (most particularly R^{1a} and/or R^{1b} do not represent —C(O)OR^{5c});

R^{1a} and R^{1b} independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g}S(O)₂N(R^{6f})R^{7f}, —OR^{5h}, —OC(O)N(R^{6g})R^{7g}, —OS(O)₂R⁵ⁱ, —OC(O)R⁵ⁿ, —OC(O)OR^{5p} or —OS(O)₂N(R⁶ⁱ)R⁷ⁱ;

for example when R^{1a} and/or R^{1b} represents Z^{2a}, then Z^{2a} preferably represents —R^{5a}, —C(O)N(R^{6a})R^{7a} or —S(O)ₘR^{5j};

for example when Z^{2a} represents —R^{5a}, then R^{5a} preferably represents C₁₋₆ alkyl optionally substituted by one or more substituents selected from ═O or, preferably, halo, —CN, —N₃, —N(R^{8b})R^{8c}, —S(O)ₙR^{8d}, —S(O)₂N(R^{8e})R^{8f} and —OS(O)₂N(R^{8g})R^{8h};

for example when Z^{2a} represents —R^{5a}, then R^{5a} preferably does not represent C₁₋₆ alkyl substituted by more than one substituent, in which the substituents include both: —OR^{8a} and fluoro; and ═O and fluoro;

for example when Z^{2a} represents —R^{5a}, R^{5a} represents C₁₋₆ alkyl substituted by one or more substituents, in which at least one of the substituents is —OR^{8a}, then preferably, R^{8a} represents C₁₋₆ alkyl optionally substituted as hereinbefore defined;

R^{1a} and R^{1b} independently represent —S(O)ₘR^{5j}, or, preferably, hydrogen, —C(O)N(R^{6a})R^{7a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —OC(O)R⁵ⁿ, —OC(O)OR^{5p} or —OS(O)₂N(R⁶ⁱ)R⁷ⁱ.

Further compounds of the invention that may be mentioned include those in which, for example, when ring A represents ring I), E^{a1} and E^{a4} respectively represent —C(R^{2b})═ and —C(R^{2d})═, then:

R^{2b} and/or R^{2d} do not represent —C(O)OR^{5c}, —N(R^{5k})S(O)₂R^{5m}, —C(H)(CF₃)OH, —C(O)CF₃, —C(OH)₂CF₃, —C(CF₃)₂OH or —S(O)₂N(R^{6h})R^{7h} (most particularly R^{2b} and/or R^{2d} do not represent —C(O)OR^{5c});

R^{2b} and R^{2d} independently represent hydrogen, a group selected from Z^{2a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —OC(O)R⁵ⁿ, —OC(O)OR^{5p} or —OS(O)₂N(R⁶ⁱ)R⁷ⁱ;

for example when R^{2b} and/or R^{2d} represents (X¹, and X¹ represents) Z^{2a}, then Z^{2a} preferably represents —R^{5a}, —C(O)N(R^{6a})R^{7a} or —S(O)ₘR^{5j};

for example when Z^{2a} represents —R^{5a}, then R^{5a} preferably represents C₁₋₆ alkyl optionally substituted by one or more substituents selected from ═O or, preferably, halo, —CN, —N₃, —N(R^{8b})R^{8c}, —S(O)ₙR^{8d}, —S(O)₂N(R^{8e})R^{8f} and —OS(O)₂N(R^{8g})R^{8h};

for example when Z^{2a} represents —R^{5a}, then R^{5a} preferably does not represent C₁₋₆ alkyl substituted by more than one substituent, in which the substituents include both: —OR^{8a} and fluoro; and ═O and fluoro;

for example when Z^{2a} represents —R^{5a}, R^{5a} represents C₁₋₆ alkyl substituted by one or more substituents, in which at least one of the substituents is —OR^{8a}, then preferably, R^{8a} represents C₁₋₆ alkyl optionally substituted as hereinbefore defined;

R^{2b} and R^{2d} independently represent —S(O)ₘR^{5j}, or, preferably, hydrogen, —C(O)N(R^{6a})R^{7a}, halo, —CN, —N(R^{6b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —OC(O)R⁵ⁿ, —OC(O)OR^{5p} or —OS(O)₂N(R⁶ⁱ)R⁷ⁱ.

Further compounds of the invention that may be mentioned include those in which in which, for example, when D₁, D₂ and D₃ respectively represent —C(R^{1a})═, —C(R^{1b})═ and —C(R^{1c})═; ring A represents ring (I) and E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, then:

L¹ represents a single bond, C₁₋₆ alkylene in which any one of the carbon atoms is interrupted by Q, or C₁₋₆ alkylene in which any one of the carbon atoms is replaced with —C(O)— or —C(R^{y1})(R^{y2})—;

R^{5a} represents, on each occasion when used herein, C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} or —OS(O)₂N(R^{8g})R^{8h};

R^{5a} represents, on each occasion when used herein, C₁₋₆ alkyl optionally substituted by one or more substituents selected from halo, —CN, —N₃, ═O, —N(R^{8b})R^{8c}, —S(O)ₙR^{8d}, —S(O)₂N(R^{8e})R^{8f} or —OS(O)₂N(R^{8g})R^{8h};

(e.g. one of) L² and L³ independently represent(s) a spacer group selected from —(CH₂)ₚ—C(R^{y3})(R^{y4})—(CH₂)_{q}-A¹⁶-, —(CH₂)ₚ—C(O)A¹⁷-, —(CH₂)ₚ—S—, —(CH₂)ₚ—SC(R^{y3})(R^{y4})—, —(CH₂)ₚ—S(O)₂A¹⁸-, —(CH₂)ₚ—N(R^{w})A¹⁹- or —(CH₂)ₚ—OA²⁰-;

(e.g. one of) Y² and Y³ represent(s) an aryl group optionally substituted as defined herein.

Further compounds of the invention that may be mentioned include those in which, for example, when D₁, D₂ and D₃ respectively represent —C(R^{1a})═, —C(R^{1b})═ and —C(R^{1c})═; ring A represents ring (I); and E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, then:

when R^{1a}, R^{1b}, R^{1c} or, if present, X¹ represent —N(R^{5d})C(O)R^{6c}, and R^{6c} represents R^{5a}, then R^{5a} represents a linear or branched C₁₋₆ alkyl group optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} or —OS(O)₂N(R^{8g})R^{8h};

R^{1a}, R^{1b} and R^{1c} independently represent hydrogen, a group selected from Z^{2a}, halo, —CN, —N(R^{6b})R^{7b}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;

X¹, X² and X³ independently represent a group selected from Z^{2a}, halo, —CN, —N(R^{6b})R^{7b}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ.

Yet further compounds of the invention that may be mentioned include those in which:

when, for example, ring A represents ring (I); L² or L³ represent —N(R^{w})A¹⁹-; A¹⁹ represents a single bond; and/or R^{w} represents H, then:

Y² or Y³ (as appropriate) do not represent a benzimidazolyl (such as one attached to the L² or L³ group via the imidazolyl moiety, e.g. benzimidazol-2-yl) group; when Y² or Y³ represents heteroaryl, then it is preferably a monocyclic heteroaryl group or a bicyclic heteroaryl group containing 1 to 4 heteroatoms consisting of 1, 3 or 4 nitrogen heteroatoms, 1 or 2 oxygen heteroatoms and/or 1 sulfur atom, for instance, the bicyclic heteroaryl group may contain 1 nitrogen, oxygen or sulfur heteroatom (all of which are optionally substituted by one or more substituents selected from A);

when Y² or Y³ represents a polycyclic (e.g. bicyclic) heteroaryl group, then it is preferably not attached to the L² or L³ group via a ring containing a heteroatom;

Y² and/or Y³ (as appropriate) represent(s) aryl or a 5- or 6-membered monocyclic ring (all of which are optionally substituted by one or more substituents selected from A).

Further compounds of the invention that may be mentioned include those in which ring A does not represent a triazinyl ring. That is ring A does not represent ring (I) in which E^{a1}, E^{a3} and E^{a5} all represent —N═.

Further compounds of the invention that may be mentioned include those in which for example when either L² or L³ represent —C(O)N(H)—, then Y² or Y³ (as appropriate) do not represent a tricyclic heteroaryl group (e.g. dibenzothiophene).

Further compounds of the invention that may be mentioned include those in which for example when there is an X¹, X², R^{z1}, X³ or R^{z2} substituent present, then:

X¹, X², R^{z1}, X³ or R^{z2} do not represent —C(O)N(R^{6a})R^{7a}, in which R^{6a} and R^{7a} represent R^{5a} and R^{5a} represents C₁₋₆ alkyl (e.g. ethyl) terminally substituted with a ═O group (so forming an aldehyde);

for example when R^{6a} and/or R^{7a} represent R^{5a}, then R^{5a} represents C₁₋₆ alkyl optionally substituted by one or more substituents selected from halo, —CN, —N₃, —OR^{8a}, —N(R^{8b})R^{8c}, —S(O)ₙR^{8d}, —S(O)₂N(R^{8e})R^{8f} and/or —OS(O)₂N(R^{8g})R^{8h}.

Preferred compounds of the invention include those in which:

one (e.g. D₁, D₂ (e.g. D₂ₐ) or D₃) or none of D₁, D₂ and D₃ represent —N═;

D₁, D₂ and D₃ respectively represent —C(R^{1a})═, —C(R^{1b})═ and —C(R^{1c})═;

R^{1a}, R^{1b} and R^{1c} independently represent a group selected from Z^{2a}, —N(R^{5d})C(O)R^{6c}, —N₃, —N(R^{5k})S(O)₂R^{5m}, preferably, halo, —CN, —N(R^{6b})R^{7b}, —NO₂, —OR^{5h}, or, more preferably, hydrogen;

when ring A represents ring (I), then two (e.g. E^{a1} and E^{a2}), preferably, one (e.g. E^{a1} or E^{a2}) or, e.g. more preferably, none of E^{a1}, E^{a2}, E^{a3}, E^{a4} and Ea⁵ represent a —N═ group;

E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═;

only one of R^{2a} to R^{2e}, such as only one of R^{2b}, R^{2c} and R^{2d} (e.g. R^{2b}) may represent -L^{1a}-Y^{1a};

R^{2a} and R^{2e} independently represent a substituent selected from X¹ or, more preferably, hydrogen;

when one of R^{2a} to R^{2e} (e.g. R^{2b}, R^{2c} and R^{2d}) represents -L^{1a}-Y^{1a}, then Y^{1a} is preferably 5-tetrazolyl or, more preferably, —COOR^{9b}, in which R^{9b} is preferably C₁₋₄ alkyl or H;

R^{3c} and R^{3d} independently represent F, Cl, —CH₃, —CF₃ or, more preferably, hydrogen;

for example when ring A represents ring (II) then, one of R^{3a} and R^{3b} represents a substituent X² or, more preferably, H or -L^{1a}-Y^{1a}, and the other represents the requisite -L³-Y³ group;

R^{4b} and R^{4c} independently represent F, Cl, —CH₃, —CF₃ or, more preferably, hydrogen;

for example when ring A represents ring (III) then, one of R^{4a} and, if present, R^{4d} represents a substituent X³ or, more preferably, H or -L^{1a}-Y^{1a}, and the other represents the requisite -L³-Y³ group;

when any one of R^{3a}, R^{3b}, R^{3c}, R^{3d}, R^{4a}, R^{4b}, R^{4c} or R^{4d} (e.g. R^{3a}, R^{3b}, R^{4a} or R^{4d}) represents -L^{1a}-Y^{1a}, then Y^{1a} is preferably a 5-tetrazolyl group or —COOR^{9b}, in which R^{9b} is preferably C₁₋₄ alkyl or H;

R^{1a}, R^{1b}, R^{1c} (when such R^{1a}, R^{1b} and R^{1c} groups represent a substituent, i.e. a group other than hydrogen), X¹, X² and X³ independently represent a group selected from Z^{2a}, or, halo, —CN, —N(R^{6b})R^{7b}, —N(R^{5d})C(O)R^{6c}, —N₃, —NO₂, ——OR^{5h} or —N(R^{5k})S(O)₂R^{5m} (more preferably such R^{1a}, R^{1b} and R^{1c} groups independently represent hydrogen, or a substituent selected from Z^{2a}, or, halo, —CN, —N(R^{6b})R^{7b}, N(R^{5d})C(O)R^{6c}, —OR^{5h} or —N(R^{5k})S(O)₂R^{5m}, and each X¹, X² and X³ independently represents a group selected from Z^{2a}, or, halo, —CN, —N(R^{6b})R^{7b}, —N(R^{5d})C(O)R^{6c}, —OR^{5h} or —N(R^{5k})S(O)₂R^{5m});

Z^{1a} and Z^{2a} independently represent —C(O)OR^{5c}, —C(O)N(R^{6a})R^{7a} or, preferably, —R^{5a};

when any of the pairs R^{6a} and R^{7a}, R^{6b} and R^{7b}, R^{6d} and R^{7d}, R^{6f} and R^{7f}, R^{6g} and R^{7g}, R^{6h} and R^{7h} or R⁶ⁱ and R⁷ⁱ are linked together, they form a 5- or 6-membered ring optionally substituted by F, —OCH₃ or, preferably, ═O or R^{5a}, and which ring optionally contains an oxygen or nitrogen heteroatom (which nitrogen heteroatom may be optionally substituted, for example with a methyl group, so forming e.g. —N(H)— or —N(CH₃)—);

R^{5c} and R^{5j} independently represent R^{5a};

when R^{5a}, R^{8a}, R^{8b}, R^{8d}, R^{8e} and R^{8g} represent C₁₋₆ alkyl optionally substituted by one or more halo substituents, then those halo substituents are preferably F or Cl (especially fluoro);

R^{5a} represents C₁₋₆ (e.g. C₁₋₄) alkyl optionally substituted by one or more substituents selected from Cl, —N₃, preferably, ═O, —N(R^{8b})R^{8c} and, more preferably, F and —OR^{8a};

m and n independently represent 2;

when any one of R^{8a} to R^{8h} (e.g. R^{8a}, R^{8b}, R^{8d}, R^{8e} and R^{8g}) represents C₁₋₆ alkyl substituted by halo, then preferred halo groups are fluoro and chloro (especially fluoro);

R^{8a}, R^{8b}, R^{8d}, R^{8e} and R^{8g} independently represent H or C₁₋₃ alkyl optionally substituted by one or more fluoro atoms;

R^{8c}, R^{8f} and R^{8h} independently represent H, —S(O)₂CH₃, —S(O)₂CF₃ or C₁₋₃ alkyl optionally substituted by one or more fluoro atoms, or the relevant pairs (i.e. R^{8b} and R^{8c}, R^{8e} and R^{8f} or R^{8g} and R^{8h}) are linked together as defined herein;

when R^{8b} and R^{8c}, R^{8e} and R^{8f}or R^{8g} and R^{8h} are linked together, they form a 5- or 6-membered ring, optionally substituted by one or more (e.g. one or two) substituents selected from F, ═O or —CH₃;

M¹ and M² independently represent —N(R^{15a})R^{15b} or, preferably, —CH₃ or —CF₃;

R^{11a}, R^{12a}, R^{12b}, R^{13a}, R^{14a}, R^{14b}, R^{15a} and R^{15b} independently represent —CH₂CH₃, —CF₃ (in the case of R^{11a} and R^{13e}) or, preferably, H or —CH₃;

Y¹ and Y^{1a} independently represent —N(H)S(O)₂R^{9a}, —C(O)OR^{9b}, —S(O)₂N(R^{10j})R^{9j} or 5-tetrazolyl;

when Y¹ and/or Y^{1a} represents 5-tetrazolyl, then such a group is optionally substituted at the 1(N)-position with R^{9x}, in which R^{9x} preferably, represents hydrogen, so forming an unsubstituted 5-tetrazolyl group;

when Y¹ and/or Y^{1a} represents —P(O)(OR^{9d})₂, then, preferably, one R^{9d} group represents hydrogen and the other represents an alkyl group as defined herein (so forming a —P(O)(O-alkyl)(OH) group) or, more preferably, both R^{9d} groups represent hydrogen (so forming a —P(O)(OH)₂ group);

when any pair of R^{9f} and R^{10f}, R^{9g} and R^{10g}, and R⁹ⁱ and R¹⁰ⁱ are linked together to form a 3- to 6-membered ring as hereinbefore defined, that ring is optionally substituted by one or more substituents selected from Cl, and, preferably F, ═O and/or R^{5a};

R^{9a} represents C₁₋₈ alkyl or a heterocycloalkyl group, both of which are optionally substituted by one or more substituents selected from G¹ and/or Z¹;

R^{9a} represents C₁₋₄ (e.g. C₁₋₃) alkyl optionally substituted by one or more halo (e.g. fluoro) atoms or, when D₂ₐ is D₂ and represents —N═, an aryl group (e.g. phenyl) substituted by one or more halo (e.g. fluoro or chloro) atoms;

R^{9b} to R^{9z}, R^{9aa}, R^{ab}, R^{10f}, R^{10g}, R¹⁰ⁱ and R^{10j} independently represent hydrogen or C₁₋₆ (e.g. C₁₋₄) alkyl optionally substituted by one or more halo (e.g. fluoro) atoms;

R^{9b} represents H;

R¹⁰ⁱ represents H;

R⁹ⁱ represents hydrogen or C₁₋₃ alkyl (such as methyl, ethyl and isopropyl);

A represents: aryl (e.g. phenyl) optionally substituted by B; C₁₋₈ alkyl optionally substituted by G¹ and/or Z¹; or G¹;

G¹ represents N₃, —NO₂ or, preferably, halo, cyano or -A¹-R^{16a};

A² represents a single bond or —O—;

A⁴ represents —C(O)N(R^{17d})—, —C(O)O— or, more preferably, a single bond or —C(O)—;

A⁵ represents —C(O)— or, preferably, a single bond;

Z¹ represents ═S, ═NCN, preferably, ═NOR^{16b} or, more preferably, ═O;

B represents: heteroaryl (e.g. oxazolyl, thiazolyl, thienyl or pyridyl) or, more preferably, aryl (e.g. phenyl) optionally substituted by G²; C₁₋₆ alkyl optionally substituted by G² and/or Z²; or, preferably, B represents G²;

G² represents cyano, preferably, —NO₂ or, more preferably, halo or -A⁶-R^{18a} (alternatively, G² represents cyano, or, preferably, halo or -A⁶-R^{18a});

A⁶ represents a single bond, —N(R^{19a})A⁹- or —OA¹⁰-;

A⁹ represents —C(O)N(R^{19d})—, —C(O)O— or, more preferably, a single bond or —C(O)—;

A¹⁰ represents a single bond;

Z² represents ═S, ═NCN, preferably, ═NOR^{18b} or, more preferably, ═O;

R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from hydrogen, aryl (e.g. phenyl) or heteroaryl (which latter two groups are optionally substituted by G³) or C₁₋₈ (e.g. C₁₋₆) alkyl (optionally substituted by G³ and/or Z³), or the relevant pairs are linked together as hereinbefore defined;

when any pair of R^{16a} to R^{16c} and R^{17a} to R^{17f}, or R^{18a} to R^{18c} and R^{19a} to R^{19f} are linked together, they form a 5- or 6-membered ring, optionally substituted by one or more (e.g. one or two) substituents selected from G³ and/or Z³;

G³ represents halo or -A¹¹-R^{20a};

A¹¹ represents a single bond or —O—;

A¹² represents a single bond or, preferably, —N(R^{21b})—;

A¹³ represents a single bond or, preferably, —N(R^{21c})—;

A¹⁴ and A¹⁵ independently represent a single bond, —C(O)— or —S(O)₂—;

Z³ represents ═S, ═NOR^{20b} or, preferably, ═O;

R^{20a}, R^{20b}, R^{20c}, R^{21a}, R^{21b}, R^{21c}, R^{21d}, R^{21e} and R^{21f} are independently selected from H, C₁₋₃ (e.g. C₁₋₂) alkyl (e.g. methyl) optionally substituted by one or more halo (e.g. fluoro) atoms, or optionally substituted aryl (e.g. phenyl), or the relevant pairs are linked together as defined herein;

when any pair of R^{20a} to R^{20c} and R^{21a} to R^{21f} are linked together, they form a 5- or 6-membered ring, optionally substituted by one or more (e.g. one or two) substituents selected from halo (e.g. fluoro) and C₁₋₂ alkyl (e.g. methyl);

R^{y1} and R^{y2} independently represent hydrogen or methyl, or, they are linked together to form a 3-membered cyclopropyl group;

Q represents —C(R^{y1})(R^{y2})— or —C(O)—;

L² and L³ independently represent —(CH₂)ₚ—C(R^{y3})(R^{y4})—(CH₂)_{q}-A¹⁶-, —(CH₂)ₚ—C(O)A¹⁷-, —(CH₂)ₚ—S—, —SC(R^{y3})(R^{y4})—, —(CH₂)ₚ—S(O)₂A¹⁸-, —(CH₂)ₚ—N(R^{w})A¹⁹- or —(CH₂)ₚ—O—;

A¹⁶ represents a single bond or, preferably, —C(O)—;

A¹⁸ represents —N(R^{w})— or a single bond;

A¹⁹ represents a single bond, —C(R^{y3})(R^{y4})—, —C(O)—, —C(O)C(R^{y3})(R^{y4})—, —C(O)O—, —S(O)₂— or —C(O)N(R^{w})—;

A²⁰ represents a single bond or —C(R^{y3})(R^{y4})—;

R^{y3} and R^{y4} independently represent H or X⁶, or, are linked together to form a 3-membered cyclopropyl group;

X⁴ to X⁸ independently represent C₁₋₆ (e.g. C₁₋₄) alkyl (optionally substituted by fluoro) or aryl (e.g. phenyl) optionally substituted by one or more substituents selected from halo, C₁₋₃ alkyl and —C(O)R^{26d};

R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} independently represent hydrogen or C₁₋₂ alkyl optionally substituted by ═O or, more preferably, one or more fluoro atoms;

R^{26a}, R^{26b}, R^{26c} and R^{26d} independently represent hydrogen or C₁₋₄ alkyl optionally substituted by one or more fluoro atoms.

More preferred compounds of the invention include those in which:

when ring A represents ring (I), in which there is one —N═ group present, then E^{a1}, E^{a3} or E^{a5} represents such a group;

when ring A represents ring (II), then W^{b} may represent —N(R^{3d})— (so forming a pyrrolyl or imidazolyl ring) or, more preferably, when Y^{b} represents —C(R^{3c})═, then W^{b} preferably represents —O— or, particularly, —S— (so forming a furanyl or, particularly, a thienyl ring) or when Y^{b} represents —N═, then W^{b} preferably represents —O— or —S— (so forming, for example, an oxazolyl or thiazolyl ring);

R^{3c} and R^{3d} independently represent H;

when ring A represents ring (III), then W^{c} preferably represents —N(R^{4d})—;

R^{4d} represents H;

X¹, X² and X³ independently represent halo (e.g. chloro or, especially, fluoro), —CN, —NO₂, —OR^{5h} or Z^{2a};

R^{5h} represents R^{5a};

Z^{2a} represents —R^{5a};

R^{5a} represents C₁₋₄ alkyl (such as methyl, ethyl and isopropyl) optionally substituted by one or halo (e.g. fuoro), so forming for example a difluoromethyl or trifluoromethyl group;

R^{8a}, R^{8b}, R^{8c}, R^{8d}, R^{8e}, R^{8f}, R^{8g} and R^{8h} independently represent H or C₁₋₃ alkyl optionally substituted by one or more fluoro atoms.

Preferred rings that ring A may represents include imidazolyl (e.g. 2-imidazolyl), preferably, furanyl (e.g. 2-furanyl), thienyl (e.g. 2-thienyl), oxazolyl (e.g. 2-oxazolyl), thiazolyl (e.g. 2-thiazolyl), pyridyl (e.g. 2- or 4-pyridyl), pyrrolyl (e.g. 3-pyrrolyl), imidazolyl (e.g. 4-imidazolyl) or, more preferably, phenyl. Alternatively, other preferred rings that A may represents include furanyl (e.g. 2-furanyl), thienyl (e.g. 2-thienyl), imidazolyl (e.g. 2-imidazolyl), oxazolyl (e.g. 2-oxazolyl), thiazolyl (e.g. 2-thiazolyl), or preferably pyridyl (e.g. 3-pyridyl) or phenyl.

Preferred rings that the D₁ to D₃-containing ring may represent include 2-, 3- or 4-pyridyl or, preferably, phenyl.

Preferred aryl and heteroaryl groups that Y² and Y³ may independently represent include optionally substituted (i.e. by A) phenyl, naphthyl, pyrrolyl, furanyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include pyridyl (e.g. 3-pyridyl), benzofuranyl (e.g. 5-benzofuranyl), isoquinolinyl (which may be partially saturated, for example forming 1,2,3,4-tetrahydroisoquinolinyl, e.g. 1,2,3,4-tetrahydroisoquinolin-7-yl) and, more particularly, phenyl. Alternatively, other preferred aryl and heteroaryl groups that Y² and Y³ may independently represent include optionally substituted thienyl (e.g. 2-thienyl), oxazolyl (e.g. 2-oxazolyl), thiazolyl (e.g. 2-thiazolyl), or more preferably, phenyl.

Preferred optional substituents on Y² and Y³ groups include:

—NO₂; or, more preferably,

halo (e.g. fluoro, chloro or bromo);

cyano;

C₁₋₆ alkyl, which alkyl group may be cyclic, part-cyclic, unsaturated or, preferably, linear or branched (e.g. C₁₋₄ alkyl (such as propyl (e.g. n-propyl and isopropyl), ethyl or, preferably, butyl (e.g. t-butyl or n-butyl) or methyl), all of which are optionally substituted with one or more halo (e.g. fluoro) groups (so forming, for example, fluoromethyl, difluoromethyl or, preferably, trifluoromethyl);

heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

—OR²⁶;

—SR²⁶;

—C(O)R²⁶;

—C(O)OR²⁶;

—N(R²⁶)R²⁷; and

—S(O)₂R²⁸;

wherein R²⁶ and R²⁷ independently represent, on each occasion when used herein, H, C₁₋₆ alkyl, such as C₁₋₅ (e.g. C₁₋₄) alkyl (e.g. ethyl, n-propyl, cyclopentyl, or, preferably, butyl (e.g. t-butyl or, preferably, n-butyl), cyclopropyl, methyl or isopropyl) optionally substituted by one or more halo (e.g. fluoro) groups (so forming e.g. a trifluoromethyl group) or aryl (e.g. phenyl) optionally substituted by one or more halo or C₁₋₃ (e.g. C₁₋₂) alkyl groups (which alkyl group is optionally substituted by one or more halo (e.g. fluoro) atoms); and R²⁸ preferably represents aryl or, particularly C₁₋₆ alkyl, for example as defined in respect of R²⁶ and R²⁷.

Particularly preferred compounds of the invention include those in which:

D_{2b} or, preferably, D₂ₐ represents D₂, and the other (i.e. preferably D_{2b}) represents —C(-L²-Y²);

D₁ and D₃ respectively represent —C(R^{1a})═ and —C(R^{1c})═;

D₂ represents —C(R^{1b})═ or —N═;

when R^{1a}, R^{1b} or R^{1c} represent a substituent other than hydrogen, then that substituent is preferably —OR^{5h}, —N(R^{6b})R^{7b}, —CN or, more preferably, Z^{2a} (e.g. R^{5a}, such as C₁₋₃ alkyl optionally substituted by one or more fluoro atoms) or halo (e.g. fluoro);

R^{1a}, R^{1b} and R^{1c} independently represent hydrogen or a substituent as defined herein (especially halo, e.g. fluoro);

any one of R^{1a}, R^{1b} and R^{1c} (e.g. R^{1c} or, preferably, R^{1b}) represents hydrogen or a substituent as defined herein (especially halo, e.g. fluoro), and the others represent hydrogen (most preferably R^{1a}, R^{1b} and R^{1c} independently represent hydrogen);

ring A represents ring I) as hereinbefore defined;

E^{a1} represents —C(H)═ or —N═;

E^{a2} represents —C(R^{2c})═ or —N═;

E^{a3} and E^{a4} represent —C(R^{2b})═, and —C(R^{2d})═, respectively;

E^{a5} represents —C(H)═;

one of R^{2b} or R^{2c} (preferably R^{2c}) represents the requisite -L³-Y³ group and the other represents a substituent selected from X¹ or, preferably, hydrogen or -L^{1a}-Y^{1a};

only one of E^{a1}, E^{a2}, E^{a3}, E^{a4} and E^{a5} may represent —N═ (or each of these respectively represent —C(R^{2a})═, —C(R^{2b})═, —C(^{2c})═, —C(R^{2d})═ and —C(R^{2e})═);

R^{2a} and R^{2e} independently represent hydrogen;

R^{2d} represents hydrogen;

X¹, X² and X³ independently represent —OR^{5h}, Z^{2a}, or, most preferably halo (e.g. chloro or, especially, fluoro) (e.g. X¹ represents fluoro);

L¹ and L^{1a} independently represent a single bond or C₁₋₄ (e.g. C₁₋₃) alkylene (e.g. methylene or ethylene), which alkylene group is optionally unsaturated (so forming, for example, —CH₂═CH₂—);

L¹ represents a single bond or C₁₋₄ alkylene (e.g. methylene, ethylene or ethenylene), in which any one of the carbon atoms may be replaced by —C(O)—;

L^{1a} represents a single bond;

Y¹ and Y^{1a} independently represent 5-tetrazolyl (e.g. unsubstituted 5-tetrazolyl) or, preferably, —C(O)OR^{9b} or —N(H)SO₂R^{6a};

R^{9a} represents an aryl group optionally substituted by one or more (e.g. two) halo (e.g. fluoro or chloro) atoms;

R^{9b} represents hydrogen or C₁₋₆ (e.g. C₁₋₄) alkyl (such as butyl, e.g. t-butyl, or methyl);

Y² and Y³ independently represent aryl (e.g. phenyl) or heteroaryl (e.g. a monocyclic 5- or 6-membered or a bicyclic 9- or 10-membered heteroaryl group preferably containing one to three heteroatom(s) selected from sulfur or, particularly, nitrogen or oxygen, so forming for example pyridyl, benzofuranyl or fully or partially aromatic isoquinolinyl), both of which are optionally substituted by one or more (e.g. one to three) substituents selected from A;

A represents I) C₁₋₈ (e.g. C₁₋₆) alkyl (e.g. n-butyl, t-butyl or methyl) optionally substituted by one or more substituents selected from G¹; or II) G¹;

G¹ represents —NO₂ or, more preferably, halo (e.g. fluoro or chloro), cyano or A₁-R^{16a};

A¹ represents a single bond, —C(O)A²-, —S—, —S(O)₂A³-, —N(R^{17a})A⁴- or —OA⁵-;

A², A³, A⁴ and A⁵ independently represent a single bond;

R^{16a} represents hydrogen or C₁₋₈ alkyl (such as C₁₋₆ alkyl or C₃₋₆ cycloalkyl, e.g. cyclopropyl, cyclopentyl, butyl, isopropyl, ethyl or methyl) optionally substituted by one or more groups selected from G³;

R^{17a} represents hydrogen or, preferably, C₁₋₆ (e.g. C₁₋₃) alkyl (such as methyl);

G³ represents halo (e.g. fluoro);

L² and L³ independently, represent a spacer group selected from —(CH₂)ₚ—C(O)A¹⁷-, —(CH₂)ₚ—S(O)₂A¹⁸-, —(CH₂)ₚ—N(R^{w})A¹⁹- and —(CH₂)ₚ—OA²⁰ (e.g. —(CH₂)ₚ—O—);

p represents 0 or 1;

when L² or L³ represent —(CH₂)ₚ—S(O)₂A¹⁸- , —(CH₂)ₚ—N(R^{w})A¹⁹- or —(CH₂)ₚ—O—, then p preferably represents 0;

when L² or L³ represent —(CH₂)—C(O)A¹⁷-, then p may represent 0 or 1;

A¹⁷ represents —N(R^{w})— or, preferably, —N(R^{w})SO₂—;

A¹⁸ represents —N(R^{w})—;

A¹⁹ represents a single bond, —C(R^{y3})(R^{y4})—, —C(O)—, —C(O)C(R^{y3})(R^{y4})—, —S(O)₂— or —C(O)N(R^{w})—;

R^{w} represents hydrogen or X⁸;

when A¹⁷ represents —N(R^{w})SO₂—, then R^{w} represents hydrogen;

when A¹⁹ represents —C(O)N(R^{w})—, then R^{w} represents hydrogen;

R^{y3} and R^{y4} independently represent hydrogen;

X⁶ represents C₁₋₄ alkyl (e.g. butyl or methyl) or aryl (e.g. phenyl) optionally substituted by one or more substituents selected from halo (e.g. chloro or, preferably, fluoro) and —C(O)R^{26d} (so forming for example a halophenyl or cyclopropylcarbonylphenyl group);

R^{26d} represents C₁₋₄ alkyl (e.g. cyclic C₃₋₄ alkyl such as cyclopropyl).

Particularly preferred compounds of the invention include:

D₂ₐ represents D₂;

D_{2b} represents —C(-L²-Y²)═;

D₁, D₂ and D₃ respectively represent —C(R^{1a})═, —C(R^{1b})═ and —C(R^{1c})═;

R^{1a}, R^{1b} and R^{1c} independently represent hydrogen;

ring A represents ring I);

E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═,

R^{2a}, R^{2c}, R^{2d} and R^{2e} independently represent hydrogen;

R^{2b} represents hydrogen or -L^{1a}-Y^{1a};

L¹ and L^{1a} independently represent a direct bond;

Y¹ and Y^{1a} independently represent —C(O)OR^{9b};

R^{9b} represents H or C₁₋₄ (e.g. C₁₋₂) alkyl (e.g. methyl);

L² and L³ independently represent —(CH₂)ₚ—N(R^{w})A¹⁹-;

p represents 0;

R^{w} represents hydrogen;

A¹⁹ represents, at each occurrence, a single bond or —S(O)₂—;

when L² represents —(CH₂)ₚ—N(R^{w})A¹⁹, then A¹⁹ preferably represents a single bond;

Y¹ and Y² independently represent phenyl optionally substituted by one or more substituents selected from A;

A represents halo (e.g. chloro or, preferably, fluoro) or G¹;

G¹ represents -A¹-R^{16a};

A¹ represents —OA⁵-;

A⁵ represents a single bond;

R^{16a} represents C₁₋₆ (e.g. C₁₋₄) alkyl (e.g. butyl, such as n-butyl).

Preferred Y² and Y³ groups include, e.g. when they represent aryl groups, 3,4-difluorophenyl and 4-n-butoxyphenyl.

Preferred substituents on Y² and Y³ groups include C₁₋₆ (e.g. C₁₋₄) alkyl or, preferably, halo (e.g. chloro or, preferably, fluoro) or C₁₋₆ (e.g. C₁₋₄) alkoxy (e.g. butoxy such as n-butoxy).

Specific L² and L³ groups that may be mentioned include —N(H)— and —N(H)S(O)₂—.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which L² and/or L³ represents —(CH₂)ₚ—N(R^{w})A¹⁹- in which p represents 0 and R^{w} represents H, reaction of a compound of formula II,

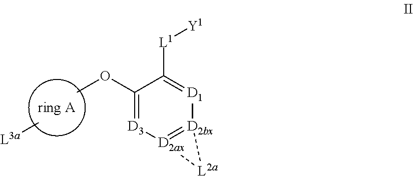

or a protected derivative thereof (e.g, an amino-protected derivative) wherein one of D₂ₐₓ and D_{2bx} represents D₂ and the other represents —C(-L^{2a})= (i.e. the L^{2a} substituent is attached to either one of D₂ₐₓ and D_{2bx}), L^{2a} represents —NH₂ or -L²-Y², L^{3a} represents —NH₂ or -L³-Y³, provided that at least one of L^{2a} and L^{3a} represents —NH₂, and ring A, D₁, D₂, D₃, L¹ and Y¹ are as hereinbefore defined, with:

(A) when A¹⁹ represents —C(O)N(R^{w})—, in which R^{w} represents H:
(a) a compound of formula III,

Y^{a}—N═C═O III
; or
(b) with CO (or a reagent that is a suitable source of CO (e.g. Mo(CO)₆ or Co₂(CO)₈)) or a reagent such as phosgene or triphosgene in the presence of a compound of formula IV,

Y^{a}—NH₂ IV

wherein, in both cases, Y^{a} represents Y² or Y³ (as appropriate/required) as hereinbefore defined. For example, in the case of (a) above, in the presence of a suitable solvent (e.g. THF, dioxane or diethyl ether) under reaction conditions known to those skilled in the art (e.g. at room temperature). In the case of (b), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). The skilled person will appreciate that for preparation of compounds of formula I in which -L²-Y² represents —C(O)N(H)-Y² and -L³-Y³ represents —C(O)N(H)—Y³ and Y² and Y³ are different, two different compounds of formula III or IV (as appropriate) will need to be employed in successive reaction steps. For the preparation of such compounds starting from compounds of formula II in which both of L^{2a} and L^{3a} represent —NH₂, then mono-protection (at a single amino group) followed by deprotection may be necessary, or the reaction may be performed with less than 2 equivalents of the compound of formula III or IV (as appropriate);

(B) when A¹⁹ represents —S(O)₂N(R^{w})—, reaction with a compound of formula V,

Y^{a}—N═S═O V

wherein Y^{a} is as hereinbefore defined, for example under reaction conditions described hereinbefore in respect of process step (i)(A)(a) above, followed by standard oxidation reaction conditions (for example, reaction in the presence of an oxidising reagent such as meta-chloroperbenzoic acid in the presence of a suitable solvent such as dichloromethane e.g. as described in Journal of Organic Chemistry, (1988) 53(13), 3012-16, or, KMnO₄, e.g. as described in Journal of Organic Chemistry, (1979), 44(13), 2055-61. The skilled person will also appreciate that the compound of formula V may need to be prepared, for example from a corresponding compound of formula IV as defined above, and SO₂ (or a suitable source thereof) or SOCl₂;

(C) when A¹⁹ represents a single bond, with a compound of formula VI,

Y^{a}-L^{a} VI

wherein L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ (or a protected derivative thereof, e.g. an alkyl protected derivative, so forming, for example a 4,4,5,5-tetramethyl-1,3,2-dioxaborolan-2-yl group) and Y^{a} is as hereinbefore defined, for example optionally in the presence of an appropriate metal catalyst (or a salt or complex thereof) such as Cu, Cu(OAc)₂, Cul (or Cul/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, Nal or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Y^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation;

(D) when A¹⁹ represents —S(O)₂—, —C(O)—, —C(R^{y3})(R^{y4})—, —C(O)—C(R^{y3})(R^{y4})— or —C(O)O—, with a compound of formula VII,

Y^{a}-A^{19a}-L^{a} VII

wherein A^{19a} represents —S(O)₂—, —C(O)—, —C(R^{y3})(R^{y4})—, —C(O)—C(R^{y3})(R^{y4})— or —C(O)O—, and Y^{a} and L^{a} are as hereinbefore defined, and L^{a} is preferably, bromo or chloro, under reaction conditions known to those skilled in the art, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine);

(ii) for compounds of formula I in which one of L² and L³ represents —N(R^{w})C(O)N(R^{w})— and the other represents —NH₂ (or a protected derivative thereof) or —N(R^{w})C(O)N(R^{w})—, in which R^{w} represents H (in all cases) reaction of a compound of formula VIII,

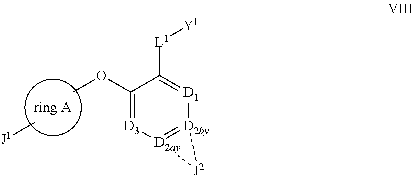

wherein one of D_{2ay} and D_{2by} represents D₂ and the other represents —C(-J²)= (i.e. the J² substituent is attached to either one of D₂ₐₓ and D_{2bx}), one of J¹ or J² represents —N═C═O and the other represents -L²-Y² or -L³-Y³ (as appropriate), —NH₂ (or a protected derivative thereof) or —N═C═O (as appropriate), and ring A, D₁, D₂, D₃, L¹ and Y¹ are as hereinbefore defined, with a compound of formula V as hereinbefore defined, under reaction conditions known to those skilled in the art, such as those described hereinbefore in respect of process step (i)(A)(b) above;

(iii) reaction of a compound of formula IX,

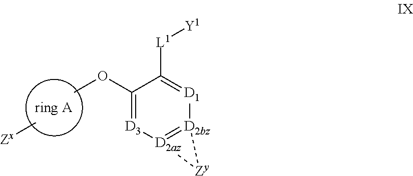

wherein one of D_{2az} and D_{2bz} represents D₂ and the other represents —C(—Z^{y})═ (i.e. the Z^{y} substituent is attached to either one of D_{2az} and D_{2bz}), Z^{x} and Z^{y} independently represent a suitable leaving group such as chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and ring A, D₁, D₂, D₃, L¹ and Y¹ are as hereinbefore defined, with a (or two separate) compound(s) (as appropriate/required) of formula X,

Y^{a}-L^{x}-H X

wherein L^{x} represents L² or L³ (as appropriate/required), and Y^{a} is as hereinbefore defined, under suitable reaction conditions known to those skilled in the art, for example such as those hereinbefore described in respect of process (i)(B) or (i)(C) above or (e.g. when L^{x} represents —S(O)₂A¹⁸-, in which A¹⁸ represents —N(R^{w})—) under Ullman reaction conditions such as those described in Tetrahedron Letters, (2006), 47(28), 4973-4978. The skilled person will appreciate that when compounds of formula I in which L² and L³ are different are required, then reaction with different compounds of formula X (for example, first reaction with a compound of formula X in which L^{x} represents —N(R^{w})A¹⁹-, followed by reaction with another, separate, compound of formula X in which L^{x} represents —OA²⁰-) may be required;

(iv) compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), may be prepared by reaction of a corresponding compound of formula I in which such a group is present that does represent hydrogen with a compound of formula XI,

R^{wy}-L^{b} XI

wherein R^{wy} represents either R^{w} (as appropriate) as hereinbefore defined provided that it does not represent hydrogen (or R^{w} represents a R⁵ to R¹⁹ group in which those groups do not represent hydrogen), and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (i)(C) above. The skilled person will appreciate that various groups (e.g. primary amino groups) may need to be mono-protected and then subsequently deprotected following reaction with the compound of formula XI;

(v) compounds of formula I in which there is a R^{w} group present that does not represent hydrogen, an aryl group or a heteroaryl group (or if there is a R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen, an aryl group or a hetereoaryl group), may be prepared by reaction of a corresponding compound of formula I in which such a group is present that does represent hydrogen with a compound of formula XII,

R^{wy}-L^{c} XII

wherein R^{wy} represents either R^{w} (as appropriate) as hereinbefore defined (e.g. R^{w} represents C₁₋₆ alkyl (optionally substituted by one or more substituents selected from halo, —CN, —N(R^{24a})R^{25a}, —OR^{24b}, ═O)) provided that it does not represent hydrogen, an aryl group or a heteroaryl group (or R^{w} represents a R⁵ to R¹⁹ group in which those groups do not represent hydrogen, an aryl group or a heteroaryl group), and L^{c} represents a suitable leaving group such as chloro, bromo, iodo, a sulfonate group (e.g. —OS(O)₂CF₃, —OS(O)₂CH₃, —OS(O)₂PhMe or a nonaflate), or a similar group known to the skilled person, under reaction conditions known to those skilled in the art, for example those hereinbefore described in respect of process step (i)(D) above;

(vi) for compounds of formula I that contain only saturated alkyl groups, reduction of a corresponding compound of formula I that contains an unsaturation, such as a double or triple bond, in the presence of suitable reducing conditions, for example by catalytic (e.g. employing Pd) hydrogenation;

(vii) for compounds of formula I in which Y¹ and/or, if present, Y^{1a} represents —C(O)OR^{9b}, —S(O)₃R^{9c}, —P(O)(OR^{9d})₂, or —B(OR^{9h})₂, in which R^{9b}, R^{9c}, R^{9d} and R^{9h} represent hydrogen (or, e.g. in the case of compounds in which Y¹ and/or Y^{1a} represent —C(O)OR^{9b}, other carboxylic acid or ester protected derivatives (e.g. amide derivatives)), hydrolysis of a corresponding compound of formula lin which R^{9b}, R^{9c}, R^{9d} or R^{9h} (as appropriate) does not represent H, or, for compounds of formula I in which Y¹ and/or, if present, Y^{1a} represents —P(O)(OR^{9d})₂ or —S(O)₃R^{9c}, in which R^{9c} and R^{9d} represent H, a corresponding compound of formula I in which Y¹ and/or Y^{1a} represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate), all under standard conditions, for example in the presence of an aqueous solution of base (e.g. aqueous 2M NaOH) optionally in the presence of an (additional) organic solvent (such as dioxane), which reaction mixture may be stirred at room or, preferably, elevated temperature for a period of time until hydrolysis is complete (e.g. 5 hours);

(viii) for compounds of formula I in which Y¹ and/or, if present, Y^{1a} represents —C(O)OR^{9b}, S(O)₃R^{9c}, —P(O)(OR^{9d})₂, —P(O)(OR^{9e})N(R^{10f})R^{f} or —B(OR^{9h})₂and R^{9b} to R^{9e} and R^{9h} (i.e. those R⁹ groups attached to an oxygen atom) do not represent H:
(A) esterification (or the like) of a corresponding compound of formula I in which R^{9b} to R^{9e} and R^{9h} represent H; or
(B) trans-esterification (or the like) of a corresponding compound of formula I in which R^{9b} to R^{9e} and R^{9h} do not represent H (and does not represent the same value of the corresponding R^{9b} to R^{9e} and R^{9h} group in the compound of formula I to be prepared),

under standard conditions in the presence of the appropriate alcohol of formula XIII,

R^{9za}OH XIII

in which R^{9za} represents R^{9b} to R^{9e} or R^{9h} (as appropriate) provided that it does not represent H, for example further in the presence of acid (e.g. concentrated H₂SO₄) at elevated temperature, such as at the reflux temperature of the alcohol of formula XIII;

(ix) for compounds of formula I in which Y¹ and/or, if present, Y^{1a} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L¹ and/or, if present, L^{1a}, are as hereinbefore defined, provided that they do not represent C₁₋₆ alkylene in which the carbon atom that is attached to ring A or the D₁ to D₃-containing ring is replaced with —O—, reaction of a compound of formula XIV,

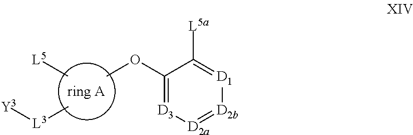

wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (or hydrogen; as appropriate), and ring A, D₁, D₂ₐ, D_{2b}, D₃, L³ and Y³ are as hereinbefore defined (the skilled person will appreciate that the compound of formula XIV in which L⁵ and/or L^{5a} represents an alkali metal (e.g. lithium), a Mg-halide or a zinc-based group may be prepared from a corresponding compound of formula XIV in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XV,

L⁶-L^{xy}-Y^{b} XV

wherein L^{xy} represents L¹ or L^{1a} (as appropriate) and Y^{b} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L⁶ represents a suitable leaving group known to those skilled in the art, such as halo (especially chloro or bromo), for example when Y^{b} represents —C(O)OR^{9b} or —S(O)₃R^{9c}, or C₁₋₃ alkoxy, for example when Y^{b} represents 13 B(OR^{9h})₂. For example, for compounds of formula I in which L¹ represents a single bond and Y¹ represents —C(O)OR^{9b}, the compound of formula XV may be Cl—C(O)OR^{9b}, The reaction may be performed under standard reaction conditions, for example in the presence of a polar aprotic solvent (e.g. THF or diethyl ether). The skilled person will appreciate that compounds of formula XIV in which L⁵ represents —B(OH)₂ are also compounds of formula I;

(x) compounds of formula I in which L¹ and/or, if present, L^{1a} represent a single bond, and Y¹ and/or, if present, Y^{1a} represent either: B(OR^{9h})₂ in which R^{9h} represents H; —S(O)₃R^{9c}; or any one of the following groups;

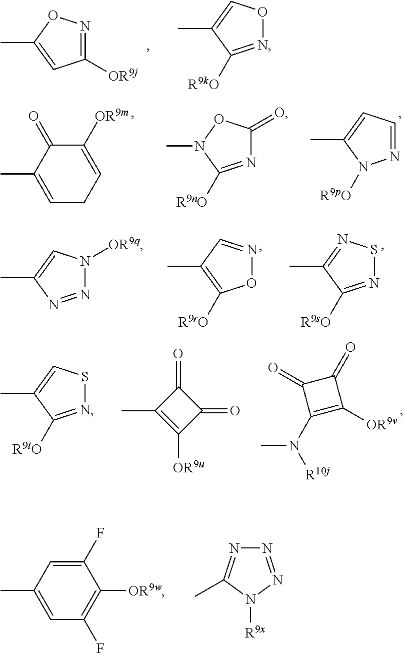

in which R^{9j}, R^{9k}, R^{9m}, R⁹ⁿ, R^{9p}, R^{9r}, R^{9s}, R^{9t}, R^{9u}, R^{9v}, R^{10j} and R^{9x} represent hydrogen, and R^{9w} is as hereinbefore defined, may be prepared in accordance with the procedures described in international patent application WO 2006/077366;

(xa) for compounds of formula I in which L¹ and/or, if present, L^{1a} represent(s) an unsubstituted 5-tetrazolyl group, reaction in accordance with procedures described in international patent application WO 2006/077366, for example, reaction of a compound corresponding to a compound of formula I, but in which the relevant L¹ and/or L^{1a} group represents —C≡N, in the presence of an appropriate reagent that effects the conversion, e.g. NaN₃, or the like, optionally in the presence of a base (such as an amine base, e.g. 1-methylpyrrolidin-2-one or the like) and an additive (such as one described herein, e.g. triethylammonium hydrochloride), for example at elevated temperature, e.g. above 80° C., such as above 100° C., e.g. about 150° C.;

(xi) compounds of formula I in which L¹ and/or, if present, L^{1a} represent a single bond, and Y¹ and/or, if present, Y^{1a} represent any one of the following groups:

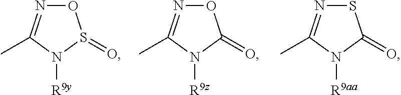

in which R^{9y}, R^{9z} and R^{9aa} represent H, may be prepared by reaction of a compound corresponding to a compound of formula I, but in which Y¹ and/or, if present, Y^{1a} represents —CN, with hydroxylamine (so forming a corresponding hydroxyamidino compound) and then with SOCl₂, R^{j}—OC(O)Cl (e.g. in the presence of heat; wherein R^{j} represents a C₁₋₆ alkyl group) or thiocarbonyl diimidazole (e.g. in the presence of a Lewis Acid such as BF₃.OEt₂), respectively, for example under reaction conditions such as those described in Naganawa et al, Bioorg. Med. Chem., (2006), 14, 7121;

(xii) compounds of formula I in which L¹ and/or, if present, L^{1a} represent a single bond, and Y¹ and/or, if present, Y^{1a} represent any one of the following groups:

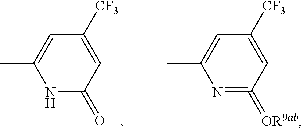

in which R^{9ab} is as hereinbefore defined, may be prepared by reaction of a compound of formula XIV wherein at least one of L⁵ and L^{5a} represents an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), and the other may represent -L¹-Y¹ or -L^{1a}-Y^{1a} (as appropriate), and ring A, D₁, D₂ₐ, D_{2b}, D₃, L³ and Y³ are as hereinbefore defined (the skilled person will appreciate that the compound of formula XIV in which L⁵ and/or L^{5a} represents an alkali metal (e.g. lithium), a Mg-halide or a zinc-based group may be prepared from a corresponding compound of formula XIV in which L⁵ and/or L^{5a} represents halo, for example under conditions such as Grignard reaction conditions, halogen-lithium exchange reaction conditions, which latter two may be followed by transmetallation, all of which reaction conditions are known to those skilled in the art), with a compound of formula XVIa or XVIb,

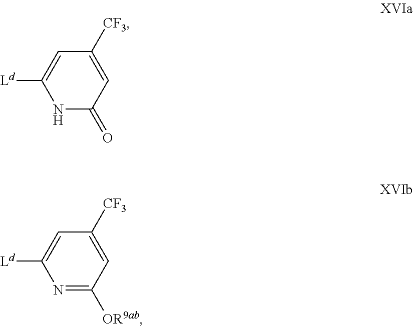

wherein R^{ab} is as hereinbefore defined and L^{d} represents (as appropriate) an appropriate alkali metal group (e.g. sodium, potassium or, especially, lithium), a —Mg-halide, a zinc-based group or a suitable leaving group such as halo or —B(OH)₂, or a protected derivative thereof (e.g. an alkyl protected derivative, so forming for example a 4,4,5,5-tetramethyl-1,3,2-dioxaborolan-2-yl group), the skilled person will appreciate that the compound of formula XVIa or XVIb in which L^{d} represents an alkali metal (e.g. lithium), a Mg-halide or a zinc-based group may be prepared from a corresponding compound of formula XVIa or XVIb in which L^{d} represents halo, for example under conditions such as Grignard reaction conditions, halogen-lithium exchange reaction conditions, which latter two may be followed by transmetallation, all of which reaction conditions are known to those skilled in the art. The reaction may be performed under standard reaction conditions, for example in the presence of a suitable solvent (e.g. THF, diethyl ether, dimethyl formamide) and, if appropriate, in the presence of a suitable catalyst (e.g. Pd(OAc)₂) and base (e.g. K₂CO₃). The skilled person will appreciate that compounds of formula XIV in which L⁵ represents —B(OH)₂ are also compounds of formula I;

(xiii) for compounds of formula I in which L¹ and/or, if present, L^{1a} represent a single bond, and Y¹ and/or, if present, Y^{1a} represent —C(O)OR^{9b} in which R^{9b} is H, reaction of a compound of formula XIV as hereinbefore defined but in which L⁵ and/or L^{5a} (as appropriate) represents either:
(I) an alkali metal (for example, such as one defined in respect of process step (ix) above); or
(II) —Mg-halide,

with carbon dioxide, followed by acidification under standard conditions known to those skilled in the art, for example, in the presence of aqueous hydrochloric acid;

(xiv) for compounds of formula I in which L¹ and/or, if present, L^{1a} represent a single bond, and Y¹ and/or, if present, Y^{1a} represent —C(O)OR^{9b}, reaction of a corresponding compound of formula XIV as hereinbefore defined but in which L⁵ and/or L^{5a} (as appropriate) is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or Co₂(CO)₈)), in the presence of a compound of formula XVII,

R^{9b}OH XVII

wherein R^{9b} is as hereinbefore defined, and an appropriate catalyst system (e.g. a palladium catalyst, such as PdCl₂, Pd(OAc)₂, Pd(Ph₃P)₂Cl₂, Pd(Ph₃P)₄, Pd₂(dba)₃ or the like) under conditions known to those skilled in the art;

(xv) reaction of either a compound of formula XVIII or XIX,

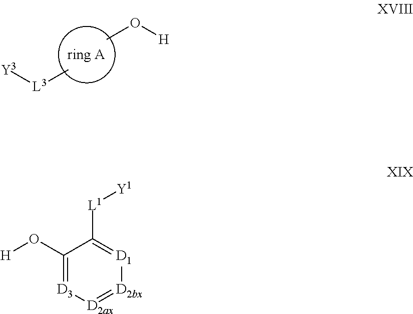

respectively with a compound of formula XX or XXI,

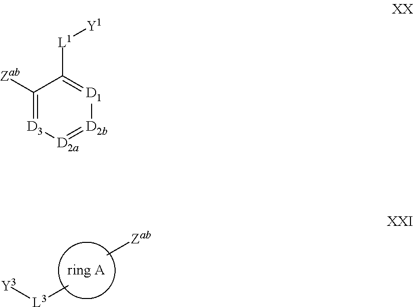

wherein (in all cases) Z^{ab} represents a suitable leaving group such as one hereinbefore defined in respect of Z^{x} or, more preferably fluoro, and ring A, D₁, D₂ₐ, D_{2b}, D₃, L¹, Y¹, L³ and Y³ are as hereinbefore defined, under standard nucleophilic aromatic substitution reaction conditions, for example in the presence of a suitable base and solvent (such as those hereinbefore defined in process step (i)(D) above);

(xvi) for compounds of formula I in which L¹ or, if present, L^{1a} represents C₁₋₆ alkylene, and Y¹ and, if present, Y^{1a} preferably represent —C(O)OR^{9b} in which R^{9b} is other than hydrogen, reaction of a compound of formula XXII

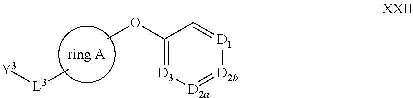

wherein ring A, D₁, D₂ₐ, D_{2b}, D₃, L³ and Y³ are as hereinbefore defined, with a compound of formula XXIII,

Z^{aa}-L^{aa}-Y^{aa} XXIII

wherein L^{aa} represents C₁₋₆ alkylene, Y^{aa} represents Y¹ (or Y^{1a}) as hereinbefore defined, but preferably —C(O)OR^{9b} in which R^{9b} is other than hydrogen, Z^{aa} represents a suitable leaving group such as one hereinbefore defined in respect of Z^{x}, and preferably represents bromo, under standard electrophilic aromatic substitution reaction conditions, e.g. in the presence of a suitable base and solvent such as those mentioned hereinbefore in respect of process step (i)(C), or optionally in the presence of a Lewis acid such as AlCl₃ under Friedel-Crafts conditions;

(xvii) for compounds of formula I in which L¹ represents —CH═CH—, reaction of a compound of formula XXIV,

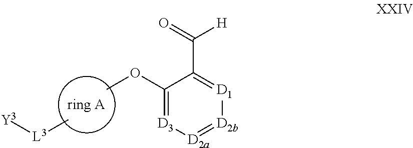

wherein ring A, D₁, D₂ₐ, D_{2b}, D₃, L³ and Y³ are as hereinbefore defined, with a compound of formula XXV,

(EtO)₂P(O)CH₂—Y¹ XXV

or the like, or a compound of formula XVI,

(Ph)₃P═CH—Y¹ XXVI

wherein (in both cases), Y¹ is as hereinbefore defined (and preferably represents —C(O)OR^{9b}, in which R^{9b} is preferably other than hydrogen), under standard Horner-Wadsworth-Emmons, or Wittig, reaction conditions, as appropriate;

(xviii) for compounds of formula I in which L² and/or L³ represent —(CH₂)ₚ—C(O)A¹⁷- in which A¹⁷ represents —N(R^{w})— or —N(R^{w})SO₂—, reaction of a corresponding compound of formula XXVII,

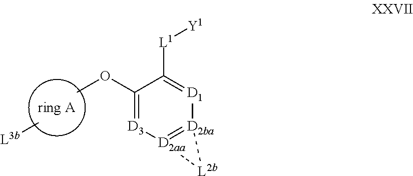

or a protected derivative thereof (e.g. an amino-protected derivative) wherein one of D₂ₐₐ and D_{2ba} represents D₂ and the other represents —C(-L^{2b}). (i.e. the L^{2b} substituent is attached to either one of D₂ₐₐ and D_{2ba}), L^{2b} represents —(CH₂)ₚ—C(O)OH or -L²-Y², L^{3b} represents —(CH₂)ₚ—C(O)OH or -L³-Y³, provided that at least one of L^{2b} and L^{3b} represents —(CH₂)ₚ—C(O)OH, and ring A, D₁, D₂, D₃, L¹ and Y¹ are as hereinbefore defined, with a compound of formula XXVIII,

H(R^{w})N-Q^{a}-Y^{a} XXVIII

wherein Q^{a} represents a direct bond or —S(O)₂—, and R^{w} and Y^{a} are as hereinbefore defined, under standard coupling reaction conditions, for example in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexa-fluorophosphate, benzotriazol-1-yloxytris-pyrrolidinophosphonium hexafluoro-phosphate, bromo-tris-pyrrolidinophosponium hexafluorophosphate, 2-(1H-benzotriazol-1-yl)-1,1,3,3-tetramethyluronium tetra-fluorocarbonate, 1-cyclohexyl-carbodiimide-3-propyloxymethyl polystyrene, O-(7-azabenzotriazol-1-yl)-N,N,N′,N′-tetramethyluronium hexafluorophosphate and/or O-benzotriazol-1-yl-N,N,N′N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof), an appropriate solvent (e.g. tetrahydrofuran, pyridine, toluene, dichloromethane, chloroform, acetonitrile, dimethylformamide, trifluoromethylbenzene, dioxane or triethylamine) and a further additive (e.g. 1-hydroxybenzotriazole hydrate). Alternatively, the carboxylic acid group of the compound of formula XXVII may be converted under standard conditions to the corresponding acyl chloride (e.g. in the presence of SOCl₂ or oxalyl chloride), which acyl chloride is then reacted with a compound of formula XXVIII, for example under similar conditions to those mentioned above;

(xix) for compounds of formula I in which L¹-Y¹ represents —C(O)N(H)SO₂R^{9a}, reaction of a corresponding compound of formula XXIX,

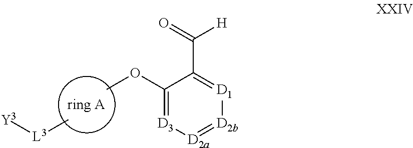

wherein ring A, D₁, D₂ₐ, D_{2b}, D₃, L³ and Y³ are as hereinbefore defined, with a compound of formula XXX,

H₂N—SO₂R^{9a} XXX

wherein R^{9a} is as hereinbefore defined, under standard coupling reaction conditions, for example such as those hereinbefore described in respect of process step (xviii) above;

(xx) for compounds of formula I in which L¹-Y¹ represents —C(O)N(H)SO₂R^{9a}, reaction of a corresponding compound of formula XXXI,

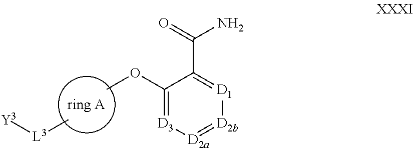

wherein ring A, D₁, D₂ₐ, D_{2b}, D₃, L³ and Y³ are as hereinbefore defined, with a compound of formula XXXII,

Cl—SO₂R^{9a} XXXII

wherein R^{9a} is as hereinbefore defined, under reaction conditions known to those skilled in the art, for example under conditions such as those hereinbefore described in respect of process step (i)(D);

(xxi) for compounds of formula I in which L² or L³ represent —N(H)—CH₂—, reductive amination of a compound of formula III as hereinbefore defined, with a compound of formula XXXIII,

Y^{a}—C(O)H XXXIII

wherein Y^{a} is as hereinbefore defined, under standard conditions, for example in the presence of a chemoselective reducing agent such as sodium triacetoxyborohydride or sodium cyanoborohydride, or alternatively, as a two-step process included condensation and then reduction, which reduction step in this instance may be performed in the presence of a stronger reducing agent such as sodium borohydride or LiAlH₄.

Compounds of formula II (or protected, e.g. mono-protected derivatives thereof) may be prepared by reduction of a compound of formula XXXIV,

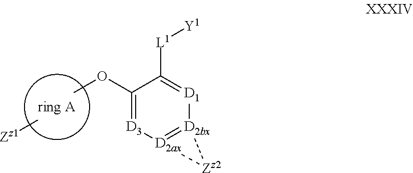

or a protected derivative thereof (e.g. an amino-protected derivative) wherein one of D₂ₐₓ and D_{2bx} represents D₂ and the other represents —C(—Z^{z2})═ (i.e. the Z^{z2} substituent is attached to either one of D₂ₐₓ and D_{2bx}), Z^{z1} represents —N₃, —NO₂, -L³-Y³ or a protected —NH₂ group, Z^{z2} represents —N₃, —NO₂, -L²-Y² or a protected —NH₂ group, provided that at least one of Z^{z1} and Z^{z2} represents —N₃ or —NO₂, and ring A, D₁, D₂, D₃, L¹ and Y¹ are as hereinbefore defined, under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane).

Compounds of formula II in which both L^{2a} and L^{3a} represent —NH₂ (or protected derivatives thereof) may also be prepared by reaction of a compound of formula IX as defined above, with ammonia, or preferably with a protected derivative thereof (e.g. benzylamine or Ph₂C═NH), under conditions such as those described hereinbefore in respect of preparation of compounds of formula I (process step (iii) above).

Compounds of formulae II or IX in which L¹ represents a single bond, and Y¹ represents —C(O)OR^{9b}, may be prepared by:

(I) reaction of a compound of formula XXXV,

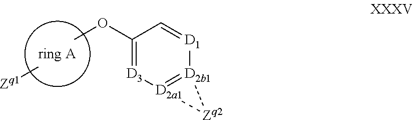

wherein Z^{q1} and Z^{q2} respectively represent Z^{x} and Z^{y} (in the case of preparation of compounds of formula IX) or L^{3a} and L^{3b} (in the case of preparation of compounds of formula III), D₂ₐ₁ and D_{2b1} respectively represent D₂ₐₓ and D_{2bx} (in the case of preparation of compounds of formula III) or D_{2az} and D_{2bz} (in the case of preparation of compounds of formula IX) and ring A, D₁, D₂ₐₓ, D_{2bx}, D_{2az}, D_{2bz}, D₃, L^{3a}, L^{3b}, Z^{x} and Z^{y} are as hereinbefore defined, with a suitable reagent such as phosgene or triphosgene in the presence of a Lewis acid, followed by reaction in the presence of a compound of formula XVII as hereinbefore defined, hence undergoing a hydrolysis or alcoholysis reaction step;

(II) for such compounds in which R^{9b} represents hydrogen, formylation of a compound of formula XXXV as hereinbefore defined, for example in the presence of suitable reagents such as P(O)Cl₃ and DMF, followed by oxidation under standard conditions;

(III) reaction of a compound of formula XXXVI,

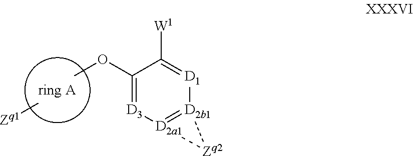

wherein W¹ represents a suitable leaving group such as one defined by Z^{x} and Z^{y} above, and ring A, D₁, D₂ₐ₁, D_{2b1}, D₃, Z^{q1} and Z^{q2} are as hereinbefore defined, are as hereinbefore defined, with CO (or a reagent that is a suitable source of CO (e.g. Mo(CO)₆ or Co₂(CO)₈) followed by reaction in the presence of a compound of formula XVII as hereinbefore defined, under reaction conditions known to those skilled in the art, for example such as those hereinbefore described in respect of preparation of compounds of formula I (process step (i)(A)(b) or (i)(C) above), e.g. the carbonylation step being performed in the presence of an appropriate precious metal (e.g. palladium) catalyst;

(IV) reaction of a compound of formula XXXVII,

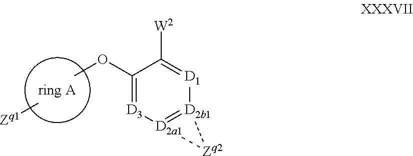

wherein W² represents a suitable group such as an appropriate alkali metal group (e.g. sodium, potassium or, especially, lithium), a —Mg-halide or a zinc-based group, and ring A, D₁, D₂ₐ₁, D_{2b1}, D₃, Z^{q1} and Z^{q2} are as hereinbefore defined, with e.g. CO₂ (in the case where R^{9b} in the compounds to be prepared represents hydrogen) or a compound of formula XV in which L^{xy} represents a single bond, Y^{b} represents —C(O)OR^{9b}, in which R^{9b} is other than hydrogen, and L⁶ represents a suitable leaving group, such as chloro or bromo or a C₁₋₁₄ (such as C₁₋₆ (e.g. C₁₋₃) alkoxy group), under reaction conditions known to those skilled in the art. The skilled person will appreciate that this reaction step may be performed directly after (i.e. in the same reaction pot) the preparation of compounds of formula) (XXVII (which is described hereinafter).

Compounds of formula II in which L^{3a} represents —NH₂, which is a to a -L^{1a}-Y^{1a} group present, which represents —C(O)OH, reaction of a compound of formula XXXVIII,

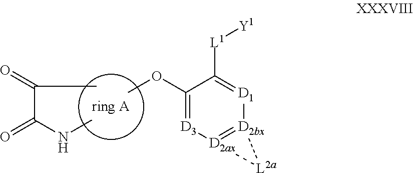

wherein ring A, D₁, D₂ₐₓ, D_{2bx}, D₃, L^{2a}, L¹ and Y¹ are as hereinbefore defined under oxidation reaction conditions, for example such as those described in Sheibley, F. E. and McNulty, J. S. J. Org. Chem., 1956; 21, 171-173, e.g. in the presence of H₂O₂, which is preferably in the presence of an alkaline solution.

Alternatively still, compounds of formula II in which D₂ₐₓ represents D₂ₐ, D_{2bx} represents —C(-L^{2a})═, L^{2a} represents —NH₂, L¹ represents a single bond and Y¹ represents —C(O)OR^{9b}, may be prepared by reaction of a compound of formula XXXIX,

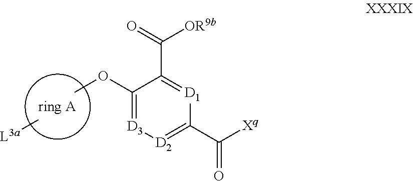

wherein X^{q} represents —OH, —NH₂ or —N₃, and L^{3a}, D₁, D₂, D₃ and ring A are as hereinbefore defined, under standard reaction conditions, for example:

(i) when X^{q} represents —OH, under Schmidt reaction conditions, or variants thereof, in the presence of HN₃ (which may be formed in by contacting NaN₃ with a strong acid such as H₂SO₄). Variants include reaction with diphenyl phosphoryl azide ((PhO)₂P(O)N₃) in the presence of an alcohol (such as tent-butanol; thereby forming a t-Boc protected derivative of formula XL) which may result in the formation of a carbamate intermediate;

(ii) when X^{q} represents —NH₂, under Hoffmann rearrangement reaction conditions, for example in the presence of NaOBr (which may be formed by contacting NaOH and Br₂) which may result in the formation of a carbamate intermediate;

(iii) when Xq represents —N₃ (which compound itself may be prepared from the corresponding acyl hydrazide under standard diazotization reaction conditions, e.g. in the presence of NaNO₂ and a strong acid such as H₂SO₄ or HCl), under Curtius rearrangement reaction conditions, which may result in the formation of an intermediate isocyanate (or a carbamate if treated with an alcohol), all of which may be followed by, if necessary (e.g. if the formation of the free amine is desired), hydrolysis, for example in the presence of water and base (e.g. one hereinbefore described in respect of process step (vii) above) when a lower alkyl carbamate (e.g. methyl or ethyl carbamate) is formed as an intermediate or under acidic conditions when e.g. a tert-butyl carbamate is formed as an intermediate, or, when a benzyl carbamate intermediate is formed, under hydrogenation reaction conditions (e.g. catalytic hydrogenation reaction conditions in the presence of a precious metal catalyst such as Pd). Similar reactants and reaction conditions may be employed for the preparation of compounds of formula III in which ring A is substituted with a —C(O)OR^{9b} group.

Compounds of formula VIII may be prepared by reaction of a corresponding compound of formula II in which L^{2a} or L^{3a} (as appropriate) represent —NH₂, with phosgene or triphosgene, for example in the presence of a suitable base (e.g. one hereinbefore defined in respect of preparation of compounds of formula I (e.g. triethylamine). When the compound of formula VIII is synthesised accordingly, it need not be isolated and/or purified when further employed in the synthesis of a compound of formula I (see process step (i) above).

Compounds of formula IX in which Z^{x} and Z^{y} represent a sulfonate group may be prepared from corresponding compounds in which the Z^{x} and Z^{y} groups represent a hydroxy group, with an appropriate reagent for the conversion of the hydroxy group to the sulfonate group (e.g. tosyl chloride, mesyl chloride, triflic anhydride and the like) under conditions known to those skilled in the art, for example in the presence of a suitable base and solvent (such as those described above in respect of process step (i)(C) or (i)(D), e.g. an aqueous solution of K₃PO₄ in toluene) preferably at or below room temperature (e.g. at about 10° C.).

Compounds of formula XXXIV in which one of Z^{z1} and Z^{z2} represents —NO₂ and the other represents -L²-Y² or -L³-Y³ (as appropriate) may be prepared by reaction of a compound of formula XVIII or XIX as hereinbefore defined, with a compound of formula XL or XLI,

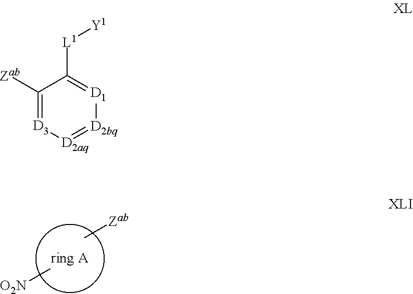

respectively, wherein one of D_{2aq} and D_{2bq} (preferably D_{2aq}) represents D₂ and the other (preferably D_{2bq}) represents —C(—NO₂)═, and Z^{ab}, D₁, D₂, D₃, D₄, L¹, Y¹ and ring A are as hereinbefore defined, under standard aromatic nucleophilic aromatic substitution reaction conditions, such as those hereinbefore described in respect of preparation of compounds of formula I (process step (xv)). The skilled person will appreciate that the presence of the nitro group, e.g. when in the para position to the Z^{ab} group will promote this reaction step due to its electron withdrawing capabilities.

Compounds of formula XXXVII may be prepared in several ways. For example, compounds of formula XXXVII in which W² represents an alkali metal such as lithium, may be prepared from a corresponding compound of formula XXXV (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamido group, or a sulfonamido group, such as an arylsulfonamido group, e.g. phenylsulfonamide), by reaction with an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)), for example in the presence of a suitable solvent, such as a polar aprotic solvent (e.g. tetrahydrofuran or diethyl ether), at sub-ambient temperatures (e.g. 0° C. to −78° C.) under an inert atmosphere. Alternatively, such compounds of formula XXXVII may be prepared by reaction of a compound of formula XXXVI in which W¹ represents chloro, bromo or iodo by a halogen-lithium reaction in the presence of an organolithium base such as t- or n-butyllithium under reaction conditions such as those described above. Compounds of formula XXXVII in which W² represents —Mg-halide may be prepared from a corresponding compound of formula XXXVI in which W¹ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XXXVII in which W² represents a zinc-based group (e.g. using ZnCl₂).

Compounds of formula XXXVIII may be prepared by reaction of a compound of formula XLII,

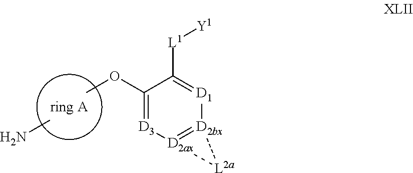

wherein ring A, D₁, D₂ₐₓ, D_{2bx}, D₃, L^{2a}, L¹ and Y¹ are as hereinbefore defined, with chloral hydrate, hydroxylamine hydrochloride, sodium sulfate and hydrochloric acid, followed by reaction in the presence of concentrated sulfuric acid, for example as described in the Sheibley et al journal article referenced herein.

Compounds of formula XXIX, or XL in which -L¹-Y¹ represents —C(O)OH, and compounds of formula XLI in which there is a -L^{1a}-Y^{1a} group present that represents —C(O)OH may be prepared by hydrolysis of a compound of formula XLIII, XLIV or XLV,

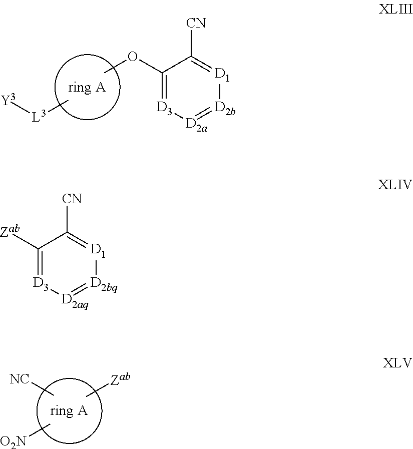

respectively, wherein Z^{ab} is as hereinbefore defined, but preferably represents fluoro or bromo, and ring A, D₁, D₂ₐ, D^{2b}, D_{2aq}, D_{2bq}, D₃, L³ and Y³ are as hereinbefore defined, under standard reaction conditions.

Compounds of formula XLIII, XLIV and XLV may be prepared by reaction of a corresponding compound of formula XLVI, XLVII or XLVIII,

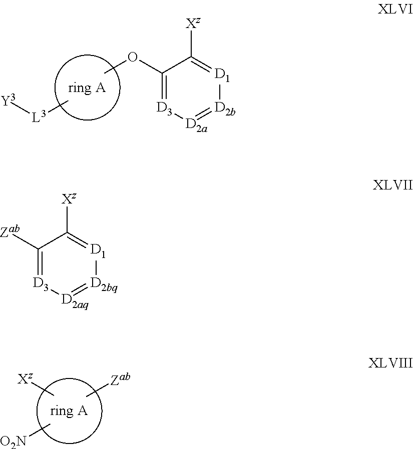

respectively, wherein X^{z} represents fluoro or bromo and ring A, D₁, D₂ₐ, D_{2b}, D_{2aq}, D_{2bq}, D₃, L³ and Y³ are as hereinbefore defined, under standard conditions, for example when X^{z} represents fluoro, in the presence of an appropriate source of cyanide ions (e.g. KCN) under standard nucleophilic aromatic substitution reaction conditions or, when X^{z} represents bromo, under palladium catalysed cyanation reaction conditions.

Compounds of formulae III, IV, V, VI, VII, X, XI, XII, XIII, XIV, XV, XVIa, XVIb, XVII, XVIII, XIX, XX, XXI, XXII, XXIII, XXIV, XXV, XXVI, XXVII, XXVIII, XXIX, XXX, XXXI, XXXII, XXXIII, XXXV, XXXVI, XXXIX, XLII, XLVI, XLVII and XLVIII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further, the compounds described herein may also be prepared in accordance with synthetic routes and techniques described in international patent application WO 2006/077366.

The substituents D₁, D₂ₐ, D_{2b}, D₃, Y¹, L³ and Y³ (as well as L² and Y²) in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where Y¹ (or, if present, Y^{1a}) represents —C(O)OR^{9b} in which R^{9b} does not initially represent hydrogen (so providing at least one ester functional group), the skilled person will appreciate that at any stage during the synthesis (e.g. the final step), the relevant R^{9b}-containing group may be hydrolysed to form a carboxylic acid functional group (i.e. a group in which R^{9b} represents hydrogen). In this respect, the skilled person may also refer to “Comprehensive Organic Functional Group Transformations” by A. R. Katritzky, O. Meth-Cohn and C. W. Rees, Pergamon Press, 1995. Other specific transformation steps include the reduction of a nitro group to an amino group, the hydrolysis of a nitrile group to a carboxylic acid group, and standard nucleophilic aromatic substitution reactions, for example in which a fluoro- or bromo-phenyl group is converted into a cyanophenyl group by employing a source of cyanide ions (e.g. KCN) as a reagent (alternatively, in this case, palladium catalysed cyanation reaction conditions may also be employed).

Further, the skilled person will appreciate that the D₁ to D₃-containing ring, as well as the A ring may be heterocycles, which moieties may be prepared with reference to a standard heterocyclic chemistry textbook (e.g. “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 or “Science of Synthesis”, Volumes 9-17 (Hetarenes and Related Ring Systems), Georg Thieme Verlag, 2006). Hence, the reactions disclosed herein that relate to compounds containing hetereocycles may also be performed with compounds that are pre-cursors to heterocycles, and which pre-cursors may be converted to those heterocycles at a later stage in the synthesis.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein. Protecting groups that may be mentioned include lactone protecting groups (or derivatives thereof), which may serve to protect both a hydroxy group and an (α-carboxy group (i.e. such that the cyclic moiety is formed between the two functional groups, for example as described hereinafter in the formation of intermediate (I)).

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

Medical and Pharmaceutical Uses

Compounds of the invention are indicated as pharmaceuticals. According to a further aspect of the invention there is provided a compound of the invention, as hereinbefore defined but without the provisos, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which Y¹ (or, if present, Y^{1a}) represents —C(O)OR^{9b} in which R^{9b} is/are other than hydrogen, so forming an ester group) may possess no or minimal pharmacological activity as such, but may be administered parenterally or orally, and thereafter be metabolised in the body to form compounds of the invention that possess pharmacological activity as such (including, but not limited to, corresponding compounds of formula I, in which Y¹ (or, if present, Y^{1a}) represents —C(O)OR^{9b} in which R^{9b} represent hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III, thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Where a condition has an inflammatory component associated with it, or a condition characterized by inflammation as a symptom, the skilled person will appreciate that compounds of the invention may be useful in the treatment of the inflammatory symptoms and/or the inflammation associated with the condition.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Loffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without the provisos, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined but without the provisos, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. thromboxane receptor (TP) antagonists, leukotriene receptor antagonists (LTRAs), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activting protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined but without the provisos; and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos, another therapeutic agent that is useful in the treatment of a respiratory disorder and/or inflammation, and a pharmaceutically-acceptable adjuvant, diluent or carrier; and

(2) a kit of parts comprising components:
(a) a pharmaceutical formulation including a compound of the invention, as hereinbefore defined but without the provisos, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier, which components (a) and (b) are each provided in a form that is suitable for administration in conjunction with the other.

The invention further provides a process for the preparation of a combination product as hereinbefore defined, which process comprises bringing into association a compound of the invention, as hereinbefore defined but without the provisos, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

Biological Tests

In Vitro Assay

In the assay, LTC₄ synthase catalyses the reaction where the substrate LTA₄ methyl ester is converted to the corresponding LTC₄ methyl ester. Recombinant human LTC₄ synthase is expressed in Piccia pastoralis and the purified enzyme is dissolved in 25 mM Tris-buffer pH 7.8 and stored at −80° C. The assay is performed in phosphate buffered saline (PBS) pH 7.4, supplemented with 5 mM glutathione (GSH). The reaction is terminated by addition of acetonitrile/MeOH/acetic acid (50/50/1). The assay is performed at rt in 96-well plates. Analysis of the formed LTC₄ methyl ester is performed with reversed phase HPLC (Waters 2795 utilizing an Onyx Monolithic C18 column). The mobile phase consists of acetonitrile/MeOH/H₂O (32.5/30/37.5) with 1% acetic acid pH adjusted with NH₃ to pH 5.6, and absorbance measured at 280 nm with a Waters 2487 UV-detector.

The following is added chronologically to each well:
1. 50 μl assay buffer, PBS with 5 mM GSH.
2. 0.5 μl inhibitor in DMSO (final conc. 1 nM-10 μM).
3. 2 μl LTC₄ synthase in PBS. The total protein concentration in this solution is 0.025 mg/ml. Incubation of the plate at room temperature for 10 minutes.
4. 1-1.5 μl LTA₄ methyl ester (final conc. 10 μM). Incubation of the plate at rt for 1 min.
5. 50 μl stop solution.

80 μl of the incubation mixture is analysed with HPLC.

Alternatively, HTRF detection can be used:

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM tris-buffer pH 7.8 supplemented with 0.1 mM glutathione (GSH) and stored at −80° C. The assay is performed in phosphate buffered saline (PBS) pH 7.4 and 5 mM GSH in 384-well plates.

The following is added chronologically to each well:

1.48 μL LTC₄ synthase in PBS with 5 mM GSH. The total protein concentration in this solution is 0.5 μg/mL.

2. 1 μL inhibitor in DMSO (final concentration 1 nM to 10 μM).

3. Incubation of the plate at room temperature for 10 min.

4. 1 μL LTA₄ (final concentration 2.5 μM).

5. Incubation of the plate at room temperature for 5 min.

6. 10 μL of the incubation mixture is analysed using homogenous time resolved fluorescent (HTRF) detection.

### EXAMPLES

The invention is illustrated by way of the following examples, in which the following abbreviations may be employed:

aq aqueous

brine saturated aqueous solution of NaCl

DMAP N,N-dimethyl-4-aminopyridine

DMF dimethylformamide

EtOAc ethyl acetate

NMR nuclear magnetic resonance

Pd₂dba₃ tris(dibenzylideneacetone)dipalladium(0)

rt room temperature

rx reflux temperature

sat saturated

XANTPHOS (9,9-dimethyl-9H-xanthene-4,5-diyl)bis(diphenylphosphine)

Chemicals specified in the synthesis of the compounds in the examples were commercially available from, e.g. Sigma-Aldrich Fine Chemicals or Acros Int.

Preparation of Starting Materials and Active Inhibitors:

### Methyl 5-bromo-2-hydroxybenzoate (I)

5-Bromo-2-hydroxybenzoic acid (2.17 g, 10 mmol) was dissolved in methanol (50 mL) and sulfuric acid (100%, 1 mL) and heated at rx for 12 h. After cooling and neutralization (NaHCO₃, aq, sat), the white precipitate was filtered off and washed with water and dried, furnishing 1.92 g (81%) of intermediate I.

### Methyl 5-bromo-2-(nitroaryloxy)benzoate (II)

I (1.9 g, 8.16 mmol), nitroaryl fluoride (9.8 mmol), K₂CO₃ (3.38 g, 24.5 mmol) and 18-crown-6 ether were dissolved in DMF (15 mL), set under inert atmosphere and stirred at rt for 24 h. After dilution (water, 200 mL) and extraction (EtOAc), the organic phase was washed with brine, dried (Na₂SO₄) and concentrated. Purification by chromatography furnished intermediate II.

### Methyl 5-(arylamino)-2-(nitroaryloxy)benzoate (III)

II (2.8 mmol), arylamine (2.8 mmol), Pd₂dba₃ (0.051 g, 0.056 mmol), XANTPHOS (0.048 g, 0.084 mmol), and Cs₂CO₃ (0.912 g, 2.8 mmol) were dissolved in toluene (10 mL) under inert atmosphere and heated at 110 ° C. with stirring for 22 h. After cooling, dilution (CH₂Cl₂) and filtration through celite the residue was concentrated and purified by chromatography to afford intermediate III.

### Methyl 2-(aminoaryloxy)-5-(arylamino)benzoate IV

III (0.773 g, 1.93 mmol) was dissolved in EtOAc (20 mL) and Pd on carbon (100 mg, 10%) was added. The mixture was stirred under hydrogen atmosphere 30 min. Filtration through celite and concentration afforded a residue which was re-crystallized from diethyl ether delivering 0.533 g, 75% of intermediate IV.

### Procedure A, Arylsulfonylation of IV

IV (0.72 mmol), arylsulfonyl chloride (1.0 mmol) and DMAP (0.017 g, 0.14 mmol) were dissolved in pyridine (5 mL) under inert atmosphere and stirred at rt for 20 h. The cooled mixture was diluted with water and extracted with EtOAc. The combined organic layers were washed with HCl (0.5 M, aq) and brine, dried (Na₂SO₄) and concentrated. The residue was purified by chromatography to afford ester V.

### Procedure B, Arylation of IV

IV (0.266 g, 0.72 mmol), aryl bromide (1.08 mmol) Pd₂dba₃ (0.013 g, 0.014 mmol), XANTPHOS (0.013 g, 0.021 mmol), and Cs₂CO₃ (0.352 g, 1.08 mmol) were dissolved in toluene (6 mL) under inert atmosphere and heated at 110° C. with stirring for 20 h. After cooling, dilution (CH₂Cl₂) and filtration through celite the residue was concentrated and purified by chromatography to afford of ester VI.

### Procedure C, Hydrolysis of V or VI Affording Va and VIa

Ester compound V or VI (0.3 mmol) was dissolved in dioxane (8 mL) and NaOH (2M, aq, 1.5 mL) and stirred at 90° C. for 30 min. The mixture was cooled, acidified with HCl (aq, 1 M) to pH=3, diluted with water (30 mL) and extracted with EtOAc. The combined organic layers were washed with water and brine and then dried (Na₂SO₄) and concentrated to afford a residue which was dissolved in diethyl ether and petroleum ether. Filtration and concentration afforded the free acid Va and VIa.

TABLE 1
Mono acid Compounds of Examples 1-4 using Procedure A or B then C.

| | | | Starting material | | Yield (%) | |
|---|---|---|---|---|---|---|
| No | Chemical name | Method | IV | Substrate | Ester | Acid |
| 1 | 2-[4-(4-Butoxy-benzene- | A | Methyl 2-(4- | 4-butoxy- | 71 | 81 |
| | sulfonylamino)- | | aminophenoxy)-5- | benzene-1- | V | Va |
| | phenoxy]-5-(3,4-difluoro- | | (3,4-difluoro- | sulfonyl | | |
| | phenyl-amino)-benzoic | | phenylamino)- | chloride | | |
| | acid | | benzoate | | | |
| 2 | 5-(3,4-Difluoro- | B | Methyl 2-(4- | 4-bromo- | 42 | 85 |
| | phenylamino)-2-[4-(3,4- | | aminophenoxy)-5- | 1,2-difluoro- | VI | VIa |
| | difluoro-phenylamino)- | | (3,4-difluoro- | benzene | | |
| | phenoxy]-benzoic acid | | phenylamino)- | | | |
| | | | benzoate | | | |
| 3 | 2-(4-Butoxy-benzene- | A | Methyl 2-amino-5- | 4-butoxy- | 67 | 54 |
| | sulfonylamino)-5-[2- | | (4-(3,4-difluoro- | benzene-1- | V | Va |
| | carboxy-4-(3,4-difluoro- | | phenyl-amino)-2- | sulfonyl | | |
| | phenylamino)-phenoxy]- | | (methoxycarbon- | chloride | | |
| | benzoic acid | | yl)-phenoxy)- | | | |
| | | | benzoate | | | |
| 4 | 2-[2-Carboxy-4-(3,4- | B | Methyl 2-amino-5- | 4-bromo- | 32 | 88 |
| | difluoro-phenylamino)- | | (4-(3,4-difluoro- | 1,2-difluoro- | VI | VIa |
| | phenoxy]-5-(3,4- | | phenylamino)-2- | benzene | | |
| | difluoro-phenylamino)- | | (methoxycarbon- | | | |
| | benzoic acid | | yl)phenoxy)- | | | |
| | | | benzoate | | | |

TABLE 2
Spectroscopic data of the compounds of Examples 1-4

| No | ¹H NMR (DMSO-d₆, 400 or 200 MHz), δ: |
|---|---|
| 1 | 12.9-12.7 (1H, br s) 9.88 (1H, s) 8.42 (1H, s) 7.64-7.52 (2H, m) 7.44 (1H, d, |
| | J = 2.9 Hz) 7.36-7.17 (2H, m) 7.06-6.68 (9H, m) 3.99 (2H, t, J = 6.4 Hz) |
| | 1.74-1.58 (2H, m) 1.48-1.28 (2H, m) 0.89 (3H, t, J = 7.3 Hz) |
| 2 | 12.9-12.8 (1H, br s) 8.40 (1H, s) 8.15 (1H, s) 7.45 (1H, d, J = 2.8 Hz) |
| | 7.36-7.12 (3H, m) 7.09-6.75 (8H, m) 6.75-6.65 (1H, m) |
| 3 | 15.6-15.0 (2H, br s) 13.2-12.4 (1H, br s) 8.40 (1H, s) 7.67-7.57 (2H, m) |
| | 7.43 (1H, d, J = 2.8 Hz)) 7.37-7.15 (4H, m) 7.04-6.75 (6H, m) 3.96 (2H, t, J = 6.5 Hz) |
| | 1.73-1.57 (2H, m) 1.48-1.28 (2H, m) 0.85 (3H, t, J = 7.3 Hz) |
| 4 | 13.3-12.6 (2H, br s) 9.3-9.1 (1H, br s) 8.43 (1H, s) 7.46 (1H, d, J = 2.8 Hz) |
| | 7.42-7.18 (6H, m) 7.13-6.92 (4H, m) 6.87-6.76 (1H, m) |

### Example 5

Title compounds of the examples were tested in the biological test described above (e.g. by HTRF detection) and were found to exhibit 50% inhibition of LTC₄ at a concentration of 10 μM or below. For example, the following representative compounds of the examples exhibited the following IC₅₀ values:

Example 1: 243 nM

Example 2: 1896 nM

Example 3: 1020 nM

Example 4: 2030 nM

1. A compound of formula I, 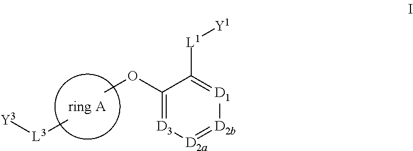
wherein
either one of D₂ₐ and D_{2b} represents D₂, and the other represents —C(-L²-Y²)═;
each of D₁, D₂ and D₃ respectively represent —C(R^{1a})═, —C(R^{1b})═ and —C(R^{1c})═, or, each of D₁, D₂ and D₃ may alternatively and independently represent —N═;
ring A represents: 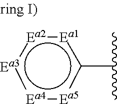
each of E^{a1}, E^{a2}, E^{a3}, E^{a4} and E^{a5} respectively represent —C(R^{2a})═, —C(R^{2b})═, —C(R^{2c})═, —C(R^{2d})═ and —C(R^{2e})═, or, each of E^{a1}, E^{a2}, E^{a3}, E^{a4} and E^{a5} may alternatively and independently represent —N═;
R^{2a} and R^{2e} independently represent hydrogen, or a substituent selected from X¹;
one of R^{2b}, R^{2c} and R^{2d} represents the requisite -L³-Y³ group, and the others independently represent hydrogen, -L^{1a}-Y^{1a} or a substituent selected from X¹; 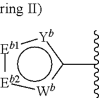
E^{b1} and E^{b2} respectively represent —C(R^{3a})═ and —C(R^{3b})═;
Y^{b} represents —C(R³c)═ or —N═;
W^{b} represents —N(R^{3d})—, —O—or —S—;
one of R^{1a}, R^{3b} and, if present, R^{3c} and R^{3d}, represents the requisite -L³-Y³ group, and the remaining R^{3a}, R^{3b} and (if present) R^{3c} substituents represents hydrogen, -L^{1a}-Y^{1a} or a substituent selected from X², and the remaining R^{3d} substituent (if present) represents hydrogen or a substituent selected from R^{z1}; or 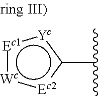
E^{c1} and E^{c2} each respectively represent —C(R^{4a})═ and —C(R^{4b})═;
Y^{c} represents —C(R^{4c})═ or —N═;
W^{c} represents —N(R^{4d})—, —O—or —S—;
one of R^{4a}, R^{4b} and, if present, R^{4c} and R^{4d} represents the requisite -L³-Y³ group, and the remaining R^{4a}, R^{4b} and (if present) R^{4c} substituents represent hydrogen, L^{1a}-Y^{1a} or a substituent selected from X³, and the remaining R^{4d} substituent (if present) represents hydrogen or a substituent selected from R^{z2};
R^{zi} and R^{z2} independently represent a group selected from Z^{1a};
R^{1a}, R^{1b} and R^{1c} independently represent hydrogen, a group selected from Z^{2a}, halo, —CN, —N(R^{8b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
X¹, X² and X³ independently represent a group selected from Z^{2a}, halo, —CN, —N(R^{8b})R^{7b}, —N(R^{5d})C(O)R^{6c}, —N(R^{5e})C(O)N(R^{6d})R^{7d}, —N(R^{5f})C(O)OR^{6e}, —N₃, —NO₂, —N(R^{5g})S(O)₂N(R^{6f})R^{7f}, —OR^{5h}, —OC(O)N(R^{6g})R^{7g}, —OS(O)₂R⁵ⁱ, —N(R^{5k})S(O)₂R^{5m}, —OC(O)R⁵ⁿ, —OC(O)OR^{5p} or —OS(O)₂N(R⁶ⁱ)R⁷ⁱ;
Z^{1a} and Z^{2a} independently represent —R^{5a}, —C(O)R^{5b}, —C(O)OR^{5c}, —C(O)N(R^{6a})R^{7a}, —S(O)ₘR^{5j} or —S(O)₂N(R^{6h})R^{7h};
R^{5b} to R^{5b}, R^{5j}, R^{5k}, R⁵ⁿ, R^{6a} to R⁶ⁱ, R^{7a}, R^{7b}, R^{7d} and R^{7f} to R⁷ⁱ independently represent H or R^{5a}; or any of the pairs R^{6a} and R^{7a}, R^{6b} and R^{7b}, R^{6d} and R^{7d}, R^{6f} and R^{7f}, R^{6g} and R^{7g}, R^{6h}, and R^{7h} or R⁶ⁱ and R⁷ⁱ may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O, —OR^{5h} and R^{5a};
R⁵ⁱ, R^{5m} and R^{5p} independently represent R^{5a};
R^{5a} represents C₁₋₆ alkyl optionally substituted by one or more substituents selected from halo, —CN, —N₃, ═O, —OR^{8a}, —N(R^{8b})R^{8c}, —S(O)ₙR^{8d}, —S(O)₂N(R^{8e})R^{8f} and —OS(O)₂N(R^{8g})R^{8h};
n represents 0, 1 or 2;
R^{8a}, R^{8b}, R^{8d}, R^{8e} and R^{8g} independently represent H or C₁₋₆ alkyl optionally substituted by one or more substituents selected from halo, ═O, —OR^{11a}, —N(R^{12a})R^{12b} and —S(O)₂-M¹;
R^{8c}, R^{8f} and R^{8b} independently represent H, —S(O)₂CH₃, —S(O)₂CF₃ or C₁₋₆ alkyl optionally substituted by one or more substituents selected from F, Cl, ═O, —OR^{13a}, —N(R^{14a})R^{14ab} and —S(O)₂—M²; or
R^{8b} and R^{8g}, R^{8e} and R^{8f} or R^{8g} and R^{8h} may be linked together to form, along with the atom(s) to which they are attached, a 3- to 6-membered ring, which ring optionally contains a further heteroatom in addition to the nitrogen atom to which these substituents are necessarily attached, and which ring is optionally substituted by one or more substituents selected from F, Cl, ═O and C₁₋₃ alkyl optionally substituted by one or more substituents selected from ═O and fluoro;
M¹ and M² independently represent —CH₃, —CH₂CH₃, —CF₃ or —N(R^{15a})R^{15b};
R^{11a} and R^{13a} independently represent H, —CH₃, —CH₂CH₃, —CF₃ or —CHF₂;
R^{12a}, R^{12b}, R^{14a}, R^{14b}, R^{15a} and R^{15b} independently represent H, —CH₃ or —CH₂CH₃;
Y¹ and Y^{1a} independently represent —N(H)SO₂R^{8a}, —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹¹ or any one of the following groups: 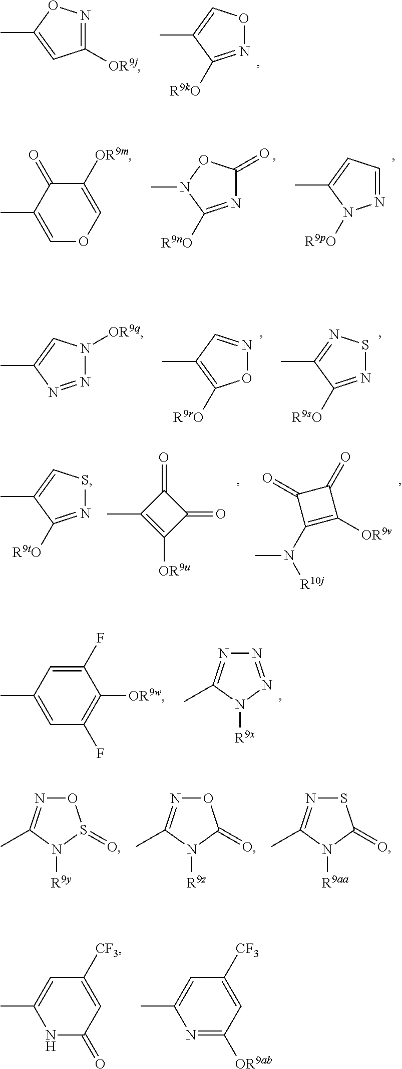
R^{9a} represents on each occasion when used herein, C₁₋₈ alkyl, a heterocycloalkyl group, an aryl group or a heteroaryl group which are optionally substituted by one or more substituents selected from G¹ and/or Z¹;
R^{9b} to R^{9z}, R^{9aa}, R^{9ab}, R^{10f}, R^{10g}, R¹⁰ⁱ and R^{10j} independently represent, on each occasion when used herein, C₁₋₈ alkyl or a heterocycloalkyl group, both of which are optionally substituted by one or more substituents selected from G¹ and/or Z¹; or
R^{9b} to R^{9z}, R^{9aa}, R^{9ab}, R^{10f}, R^{10g}, R¹⁰ⁱ and R^{10j} independently represent hydrogen; or
any pair of R^{9f} and R^{10f}, R^{9g} and R^{10g}, and R⁹ⁱ and R¹⁰ⁱ, may be linked together to form, along with the atom(s) to which they are attached, a 3- to 6-membered ring, which ring optionally contains a further heteroatom, in addition to the nitrogen atom to which these substituents are necessarily attached, and which ring is optionally substituted by one or more substituents selected from F, Cl, ═O, —OR^{5h} and R^{5a};
Y² and Y³ independently represent an aryl group or a heteroaryl group, both of which groups are optionally substituted by one or more substituents selected from A;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{16a};
wherein A¹ represents a single bond or a spacer group selected from —C(O)A²-, —S—, —S(O)ᵣA³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)ᵣ— or —S(O)ᵣN(R^{17e})—;
Z¹ represents ═O, ═S, ═NOR^{16b}, ═NS(O)₂N(R^{17f})R^{16c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{18a};
wherein A⁶ represents a single bond or a spacer group selected from —C(O)A⁷-, —S—, —S(O)ᵣA⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)ᵣ— or —S(O)ᵣN(R^{19e})—;
Z² represents ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b},R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{20a};
wherein A¹¹ represents a single bond or a spacer group selected from —C(O)A¹²-, —S—, —S(O)ᵣA¹³-, —N(R^{21a})A¹⁴- or —OA¹⁵-, in which:
A¹² represents a single bond, —O—, —N(R^{21b})— or —C(O)—;
A¹³ represents a single bond, —O—or —N(R^{21c})—;
A¹⁴ and A¹⁵ independently represent a single bond, —C(O)—, —C(O)N(R^{21d})—, —C(O)O—, —S(O)ᵣ— or —S(O)ᵣN(R^{21e})—;
Z³ represents ═O, ═S, ═NOR^{20b}, ═NS(O)₂N(R^{21f})R^{20c}, ═NCN or ═C(H)NO₂;
each r independently represents, on each occasion when used herein, 1 or 2;
R^{20a}, R^{20b}, R^{20c}, R^{21a}, R^{21b}, R^{21c}, R^{21d}, R^{21e} and R^{21f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —R^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —0R^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═0;
L¹ and L^{1a} independently represent a single bond or C₁₋₆ alkylene in which any one of the carbon atoms may be replaced by Q;
Q represents —C(R^{y1})(R^{y2})—, —C(O)— or —O—;
R^{y1} and R^{y2} independently represent H, F or X⁴; or R^{y1} and R^{y2} may be linked together to form a 3- to 6-membered ring, which ring optionally contains a heteroatom, and which ring is optionally substituted by one or more substituents selected from F, Cl, ═O and X⁵;
L² and L³ independently represent a single bond or a spacer group selected from —(CH₂)ₚ—C(R^{y3})(R^{y4})—(CH₂)_{q}-A¹⁶-, —(CH₂)ₚ—C(O)A¹⁷-, —(CH₂)ₚ—S—, —(CH₂)ₚ—SC(R^{y3})(R^{y4})—, —(CH₂)ₚ—S(O)A²¹-, —(CH₂)—S(O)₂A¹⁸-, —(CH₂)ₚ—N(R^{w})A¹⁹- or —(CH₂)—OA²⁰-, in which:
A¹⁶ represents a single bond, —O—, —N(R^{w})—, —C(O)—, or —S(O)ₘ—;
A¹⁷, A¹⁸ and A²¹ independently represent a single bond, —C(R^{y3})(R^{y4})—, —O—, —N(R^{w})— or —N(R^{w})SO₂—;
A¹⁹ and A²⁰ independently represent a single bond, —C(R^{y3})(R^{y4})—, —C(O)—, —C(O)C(R^{y3})(R^{y4})—, —C(O)N(R^{w})—, —C(O)O—, —S(O)₂— or —S(O)₂N(R^{w})—;
p and q independently represent 0, 1 or 2;
m represents 0, 1 or 2;
R^{y3} and R^{y4} independently represent H, F or X⁶; or
R^{y3} and R^{y4} may be linked together to form a 3- to 6-membered ring, which ring optionally contains a heteroatom, and which ring is optionally substituted by one or more substituents selected from F, Cl, ═O and X⁷;
R^{w} represents H or X⁸;
X⁴ to X⁸ independently represent C₁₋₆ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d})), aryl or heteroaryl (which latter two groups are optionally substituted by one or more substituents selected from halo, C₁₋₄ alkyl (optionally substituted by one or more substituents selected from fluoro, chloro and ═O, —N(R^{26a})R^{26b}, —OR^{26c} and —C(O)R^{26d});
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a}, R^{25b}, R^{26a}, R^{26b}, R^{26c} and R^{26d} are independently selected from hydrogen and C₁₋₄ alkyl, which latter group is optionally substituted by one or more substituents selected from fluoro, chloro and ═O,
or a pharmaceutically-acceptable salt thereof,
provided that when L¹ represents a direct bond; Y¹ represents —C(O)OH; ring A represents ring I):
(I) D₁, D₂ₐ and D₃ all represent —C(—COOH)═; D_{2b} represents —C(-L²-Y²)═; E^{a1}, E^{a2}, E^{a4} and E^{a5} all represent —C(H)═; E^{a3} represents —C(R^{2c})═; R^{2c} represents the requisite -L³-Y³ group; L² represents —O—; Y² represents phenyl substituted in the 4-position by A; A represents phenyl substituted in the 4-position by G²; L³ represents a direct bond; Y³ represents phenyl substituted in the 4-position by A; A represents G¹, then G¹ and G² do not both represent dodecyloxy, decyloxy, octyloxy or hexyloxy;
(II) D₁ and D₃ both represent —C(H)═; D₂ₐ represents —C(—COOH)═; D_{2b} represents —C(-L²-Y²)═; E^{a1}, E^{a4} and E^{a5} all represent —C(H)═; L² represents —O—:
(a) Y² represents phenyl substituted in the 3-position by —O—CH₂-phenyl and in the 4-position by —NO₂; E^{a3} represents —C(NO₂)═; E^{a2} represents —C(R^{2b})═; R^{2b} represents the requisite -L³-Y³ group; L³ represents —OCH₂—, then Y³ does not represent unsubstituted phenyl;
(b) Y² represents phenyl substituted in the 4-position by —S(O)₂-phenyl; E^{a2} represents —C(H)═; E^{a3} represents —C(R^{2c})═; R^{2c} represents the requisite -L³-Y³ group; L³ represents —S(O)₂—, then Y³ does not represent unsubstituted phenyl;
(III) D₁ and D₃ both represent —C(OH)═; D₂ₐ represents —C(—COOH)═; D_{2b} represents —C(-L²-Y²)═; L² represents —O—; E^{a1}, E^{a2}, E^{a4} and E^{a5} all represent —C(H)═; E^{a3} represents —C(R^{2c})═; R^{2c} represents the requisite -L³-Y³ group; L³ represents a single bond, then:
(a) Y³ does not represent unsubstituted phenyl when Y² represents (4-phenyl)phenyl;
(b) Y³ does not represent 4-hydroxyphenyl when Y² represents [(4-hydroxy)phenyl]phenyl;
(IV) D₁, D₂ₐ and D₃ all represent —C(H)═; D_{2b} represents —C(-L²-Y²)═; E^{a2} E^{a4} and E^{a5} all represent —C(H)═; E^{a3} represents —C(R^{2c})═; R^{2c} represents the requisite -L³-Y³ group; L² and L³ both represents —C(CH₃)₂—; then Y² and Y³ do not both represent 4-hydroxyphenyl when:
(a) E^{a1} represents —C(H)═;
(b) E^{al} represents —C(-L^{1a}-Y¹)═ and -L^{1a}-Y^{1a} represents —COOH.

2. A compound as claimed in claim 1, wherein D₁, D₂ and D₃ respectively represent —C(R^{1a})═, —C(R^{1b})═ and —C(R^{1c})═.

3. A compound as claimed in claim 1, wherein ring A represents ring (I).

4. A compound as claimed in claim 1, wherein E^{a1} and E^{a5} independently represent —C(H)═ and E^{a2}, E^{a3} and E^{a4} respectively represent —C(R^{2b})═, —C(R^{2c})═ and —C(R^{2d})═.

5. A compound as claimed in claim 1, wherein one of R^{2b} or R^{2c} represents the requisite -L³-Y³ group and the other represents hydrogen or -L^{1a}-R^{1a}.

6. A compound as claimed in claim 1, wherein R^{2d} represents hydrogen.

7. A compound as claimed in claim 1, wherein L¹ and L^{1a} independently represent a single bond or C₁₋₄ alkylene.

8. A compound as claimed in claim 1, wherein Y¹ and Y^{1a} independently represent —C(O)OR^{9b}.

9. A compound as claimed in claim 1, wherein R^{9b} represents C₁₋₆ alkyl or H.

10. A compound as claimed in claim 1, wherein A represents I) C₁₋₈ alkyl optionally substituted by one or more substituents selected from G¹; or II) G¹.

11. A compound as claimed in claim 1, wherein G¹ represents halo (e.g. fluoro or chloro), cyano, —NO₂ or -A¹-R^{16a}.

12. A compound as claimed in claim 1, wherein A¹ represents a single bond, —C(O)A²-, —S—, —S(O)₂A³-, —N(R^{17a})A⁴- or —OA⁵-.

13. A compound as claimed in claim 1, wherein L² and L³ independently represent a spacer group selected from —(CH₂)ₚ—C(O)A¹⁷-, —(CH₂)ₚ—S(O)₂A¹⁸-, —(CH₂)ₚ—N(R^{w})A¹⁹- and —(CH₂)ₚ—O—.

14. A compound as claimed in claim 1, wherein A¹⁷ represents —N(R^{w})SO₂—; A¹⁸ represents —N(R^{w})—; and/or A¹⁹ represents a single bond, —C(R^{y3})(R^{y4})—, —C(O)—, —C(O)C(R^{y3})(R^{y4})—, —S(O)₂— or —C(O)N(R^{w})—.

15. A compound as claimed in claim 1, wherein R^{w} represents hydrogen or X⁸.

16. A compound as claimed in claim 1, wherein X⁸ represents C₁₋₄ alkyl or aryl optionally substituted by one or more substituents selected from halo and —C(O)R^{26d}, in which R^{26d} represents C₁₋₄ alkyl.

17. A compound as claimed in claim 1, wherein Y² and Y³ independently represent optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group.

18. A compound as claimed in claim 17, wherein Y² and Y³ independently represent optionally substituted pyridyl, benzofuranyl, isoquinolinyl and/or phenyl.

19. A compound as claimed in claim 17, wherein the optional substituents are selected from halo; cyano; —NO₂; C₁₋₆ alkyl optionally substituted with one or more halo groups; heterocycloalkyl optionally substituted by one or more substituents selected from C₁₋₃ alkyl and ═O; —OR²⁶; —SR²⁶; —C(O)R²⁶; —C(O)OR²⁶; —N(R²⁶)R²⁷; and —S(O)₂R²⁸; wherein R²⁶ and R²⁷ independently represent H, C₁₋₆ alkyl optionally substituted by one or more halo groups or aryl optionally substituted by one or more halo or C₁₋₃ alkyl groups (which alkyl group is optionally substituted by one or more halo atoms); and R²⁸ represents aryl or C₁₋₆ alkyl.

20. A compound as defined in claim 1, without the provisos, or a pharmaceutically-acceptable salt thereof, for use as a pharmaceutical.

21. A pharmaceutical formulation including a compound as defined in claim 1, out the provisos, or a pharmaceutically-acceptable salt thereof, in admixture with a pharmaceutically acceptable adjuvant, diluent or carrier.

22. A compound as defined in claim 1 but without the provisos, or a pharmaceutically acceptable salt thereof, for use in the treatment of a disease in which inhibition of the synthesis of leukotriene C₄ is desired and/or required.

23. Use of a compound of formula I, as defined in claim 1 but without the provisos, or a pharmaceutically acceptable salt thereof, for the manufacture of a medicament for the treatment of a disease in which inhibition of the synthesis of leukotriene C₄ is desired and/or required.

24. A compound as claimed in claim 22, wherein the disease is a respiratory disease, inflammation and/or has an inflammatory component.

25. A compound or use as claimed in claim 24 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anemia, hypereosinofilic syndrome, or a malignancy.

26. A compound or use as claimed in claim 25, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

27. A method of treatment of a disease in which inhibition of the synthesis of leukotriene C₄ is desired and/or required, which method comprises administration of a therapeutically effective amount of a compound of formula I as defined in claim 1, or a pharmaceutically-acceptable salt thereof, to a patient suffering from, or susceptible to, such a condition.

28. A combination product comprising:
(A) a compound of formula I as defined in claim 1 but without the provisos, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

29. A combination product as claimed in claim 28 which comprises a pharmaceutical formulation including a compound of formula I as defined in claim 1, but without the provisos, or a pharmaceutically-acceptable salt thereof, another therapeutic agent that is useful in the treatment of a respiratory disorder and/or inflammation, and a pharmaceutically-acceptable adjuvant, diluent or carrier.

30. A combination product as claimed in claim 28 which comprises a kit of parts comprising components:
(a) a pharmaceutical formulation including a compound of formula I as defined in claim 1 but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
which components (a) and (b) are each provided in a form that is suitable for administration in conjunction with the other.

31. A process for the preparation of a compound of formula I as defined in claim 1, which process comprises:
(i) for compounds of formula I in which L² and/or L³ represents —(CH₂)ₚ—N(R^{w})A¹⁹- in which p represents 0 and R^{w} represents H, reaction of a compound of formula II, 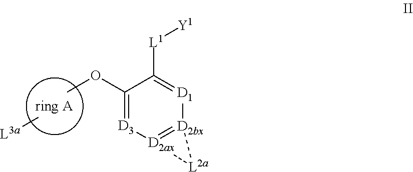
or a protected derivative thereof wherein one of D₂ₐₓ and D_{2bx} represents D₂ and the other represents —C(-L^{2a})═, L^{2a}represents —NH₂ or -L²-Y², L^{3a} represents —NH₂ or -L³-Y³, provided that at least one of L^{2a} and L^{3a} represents —NH₂, and ring A, D₁, D₂, D₃, L¹ and Y¹ are as defined in claim 1, with:
(A) when A¹⁹ represents —C(O)N(R^{w})—, in which R^{w} represents H:
(a) a compound of formula III,
Y^{a}—N═C═O III
; or
(b) with CO (or a reagent that is a suitable source of CO (e.g. Mo(CO)₆ or Co₂(CO)₈)) or a reagent such as phosgene or triphosgene in the presence of a compound of formula IV,
Y^{a}—NH₂ IV
wherein, in both cases, Y^{a} represents Y² or Y³ (as appropriate/required) as defined in claim 1,
(B) when A¹⁹ represents —S(O)₂N(R^{w})—, reaction with a compound of formula V,
Y^{a}—N═S═O V
wherein Y^{a} is as defined in claim 1;
(C) when A¹⁹ represents a single bond, with a compound of formula VI,
Y^{a}-L^{a} VI
wherein L^{a} represents a suitable leaving group and Y^{a} is as defined above;
(D) when A¹⁹ represents —S(O)₂—, —C(O)—, —C(R^{y3})(R^{y4})—, —C(O)—C(R^{y3})(R^{y4})— or —C(O)O—, with a compound of formula VII,
Y^{a}-A^{19a}-L^{a} VII
wherein A^{19a} represents —S(O)₂—, —C(O)—, —C(R^{y3})(R^{y4})—, —C(O)—C(R^{y3})(R^{y4})— or —C(O)O—, and Y^{a} and L^{a} are as defined above;
(ii) for compounds of formula I in which one of L² and L³ represents —N(R^{w})C(O)N(R^{w})— and the other represents —NH₂ (or a protected derivative thereof) or —N(R^{w})C(O)N(R^{w})—, in which R^{w} represents H (in all cases), reaction of a compound of formula VIII, 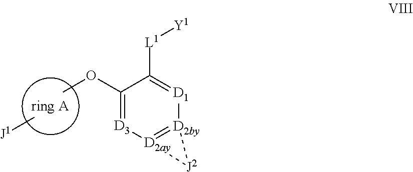
wherein one of D_{2ay} and D_{2by} represents D₂ and the other represents —C(-J²)=, one of J¹ or J² represents —N═C═O and the other represents -L²-Y² or -L³-Y³ (as appropriate), —NH₂ (or a protected derivative thereof) or —N═C═O (as appropriate), and ring A, D₁, D₂, D₃, L¹ and Y¹ are as defined in claim 1;
(iii) reaction of a compound of formula IX, 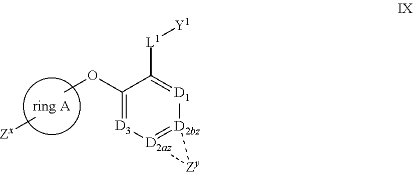
wherein one of D_{2az} and D_{2bz} represents D₂ and the other represents —C(—Z^{y})═, Z^{x} and Z^{y} independently represent a suitable leaving group, and ring A, D₁, D₂, D₃, L¹ and Y¹ are as defined in claim 1, with a (or two separate) compound(s) (as appropriate/required) of formula X,
Y^{a}-L^{x}-H X
L^{x} represents L² or L³ (as appropriate/required), and Y^{a} is as defined in claim 1;
(iv) for compounds of formula I in which there is a R^{w} group present that does not represent hydrogen (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen), reaction of a corresponding compound of formula I in which such a group is present that does represent hydrogen with a compound of formula XI,
R^{wy}-L^{b} XI
wherein R^{wy} represents either R^{w} (as appropriate) as defined in claim 1 provided that it does not represent hydrogen (or R^{w} represents a R⁵ to R¹⁹ group in which those groups do not represent hydrogen), and L^{b} represents a suitable leaving group;
(v) for compounds of formula I in which there is a R^{w} group present that does not represent hydrogen, an aryl group or a heteroaryl group (or if there is R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ or R²⁶ group present, which is attached to a heteroatom such as nitrogen or oxygen, and which does/do not represent hydrogen, an aryl group or a hetereoaryl group), by reaction of a corresponding compound of formula I in which such a group is present that does represent hydrogen with a compound of formula XII,
R^{wy}-L^{c} XII
R^{wy} represents either R^{w} (as appropriate) as defined in claim 1, provided that it does not represent hydrogen, an aryl group or a heteroaryl group (or R^{w} represents a R⁵ to R¹⁹ group in which those groups do not represent hydrogen, an aryl group or a heteroaryl group), and L^{c} represents a suitable leaving group;
(vi) for compounds of formula I that contain only saturated alkyl groups, reduction of a corresponding compound of formula I that contains an unsaturation;
(vii) for compounds of formula I in which Y¹ and/or, if present, Y^{1a} represents —C(O)OR^{9b}, —S(O)₃R^{9c}, —P(O)(OR^{9d})₂, or —B(OR^{9h})₂, in which R^{9b}, R^{9c}, R^{9d} and R^{9h} represent hydrogen, hydrolysis of a corresponding compound of formula I in which R^{9b}, R^{9c}, R^{9d} or R^{9h} (as appropriate) does not represent H, or, for compounds of formula I in which Y¹ and/or, if present, Y^{1a}, represents —P(O)(OR^{9d})₂ or S(O)₃R^{9c}, in which R^{9c} and R^{9d} represent H, a corresponding compound of formula I in which Y¹ and/or Y^{1a} represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate);
(viii) for compounds of formula I in which Y¹ and/or, if present, Y^{1a} represents —C(O)OR^{9b}, S(O)₃R^{9c}, —P(O)(OR^{9d})₂, —P(O)(OR^{9e})N(R^{10f})R^{9f} or —B(OR^{9h})₂ and R^{9b} to R^{9e} and R^{9h} do not represent H:
(A) esterification (or the like) of a corresponding compound of formula I in which R^{9b} to R^{9e} and R^{9h} represent H; or
(B) trans-esterification (or the like) of a corresponding compound of formula I in which R^{9b} to R^{9e} and R^{9h} do not represent H (and does not represent the same value of the corresponding R^{9b} to R^{9e} and R^{9h} group in the compound of formula I to be prepared),
in the presence of the appropriate alcohol of formula XIII,
R^{9za}OH XIII
in which R^{9za} represents R^{9b} to R^{9e} or R^{9h} (as appropriate) provided that it does not represent H;
(ix) for compounds of formula I in which Y¹ and/or, if present, Y^{1a} represents —C(O)OR^{9b}, —S(O)₃R⁹, —P(O)(OR^{9d})₂, —P(O)(OR^{9e})N(R^{10f})R^{9f}, —P(O)(N (R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L¹ and/or, if present, L^{1a}, are as hereinbefore defined, provided that they do not represent C₁₋₆ alkylene in which the carbon atom that is attached to ring A or the D₁ to D₃-containing ring is replaced with —O—, reaction of a compound of formula XIV, 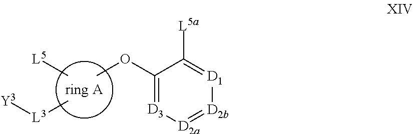
wherein at least one of L⁵ and L^{5a} represents an appropriate alkali metal group, a —Mg-halide, a zinc-based group or a suitable leaving group, and ring A, D₁, D₂ₐ, D_{2b}, D₃, L³ and Y³ are as defined in claim 1, with a compound of formula XV,
L⁶⁻L^{xy}-Y^{b} XV
wherein L^{xy} represents L¹ or L^{1a} (as appropriate) and Y^{b} represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, in which R^{9b} to R⁹ⁱ, R^{10f}, R^{10g} and R¹⁰ⁱ are other than H, and L⁶ represents a suitable leaving group;
(x) for compounds of formula I in which L¹ and/or, if present, L^{1a} represent a single bond, and Y¹ and/or, if present, Y^{1a} represent either: B(OR^{9h})₂ in which R″ represents H; —S(O)₃R^{9c}; or any one of the following groups: 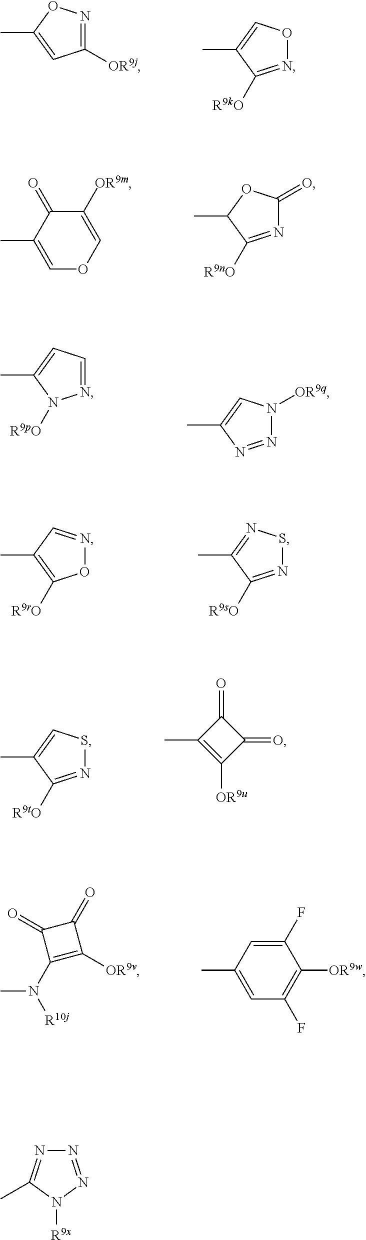
in which R^{9j}, R^{9k}, R^{9m}, R⁹ⁿ, R^{9p}, R^{9r}, R^{9s}, R^{9t}, R^{9u}, R^{9v}, R^{10j} and R^{9x} represent hydrogen, and R^{9w} is as defined in claim 1, reaction in accordance with the procedures described in international patent application WO 2006/077366;
(xa) for compounds of formula I in which L¹ and/or, if present, L^{1a} represent(s) an unsubstituted 5-tetrazolyl group, reaction of a compound corresponding to a compound of formula I, but in which the relevant L¹ and/or L^{1a} group represents —C≡N, in the presence of NaN₃, or the like;
(xi) for compounds of formula I in which L¹ and/or, if present, La represent a single bond, and Y¹ and/or, if present, Y^{1a} represent any one of the following groups: 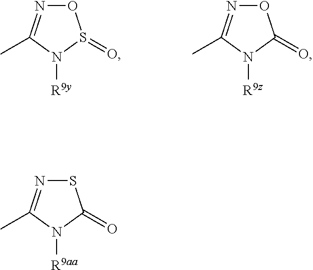
in which R^{9y}, R^{9z} and R^{9aa} represent H, reaction of a compound corresponding to a compound of formula I, but in which Y¹ and/or, if present, Y^{1a} represents —CN, with hydroxylamine and then with SOCl₂, R^{j}—OC(O)Cl (wherein R^{j} represents a C₁₋₆ alkyl group) or thiocarbonyl diimidazole;
(xii) for compounds of formula I in which L¹ and/or, if present, La represent a single bond, and Y¹ and/or, if present, Y^{1a} represent any one of the following groups: 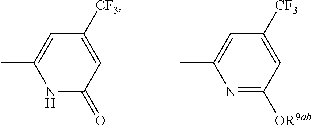
in which R^{9ab} is as defined in claim 1, reaction of a compound of formula XIV wherein at least one of L⁵ and L^{5a} represents an alkali metal group, a —Mg-halide, a zinc-based group or a leaving group, or a protected derivative thereof, and the other may represent -L¹-Y¹ or -L^{1a}-Y^{1a} (as appropriate), and ring A, D₁, D₂ₐ, D_{2b}, D₃, L³ and Y³ are as defined in claim 1, with a compound of formula XVIa or XVIb, 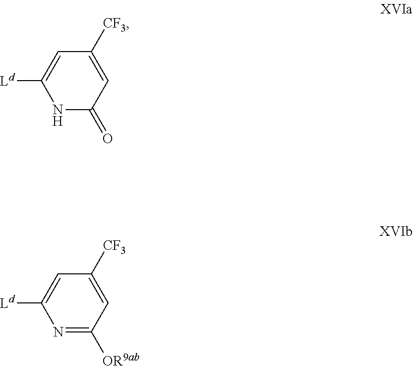
wherein R^{ab} is as defined in claim 1 and L^{d} represents (as appropriate) an alkali metal group, a —Mg-halide, a zinc-based group or a leaving group, or a protected derivative thereof;
(xiii) for compounds of formula I in which L¹ and/or, if present, La represent a single bond, and Y¹ and/or, if present, Y^{1a} represent —C(O)OR^{9b} in which R^{9b} is H, reaction of a compound of formula XIV as hereinbefore defined but in which L⁵ and/or L^{5a} (as appropriate) represents either:
(I) an alkali metal; or
(II) —Mg-halide,
with carbon dioxide, followed by acidification under standard conditions known to those skilled in the art;
(xiv) for compounds of formula I in which L¹ and/or, if present, L^{1a} represent a single bond, and Y¹ and/or, if present, Y^{1a} represent —C(O)OR^{9b}, reaction of a corresponding compound of formula XIV as defined above but in which L⁵ and/or L^{5a} (as appropriate) is a suitable leaving group with CO (or a reagent that is a suitable source of CO), in the presence of a compound of formula XVII,
R^{9b}OH XVII
wherein R^{9b} is as defined above;
(xv) reaction of either a compound of formula XVIII or XIX, 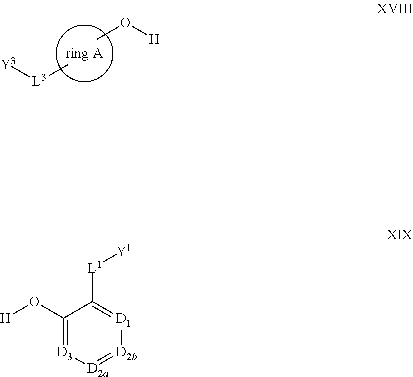
respectively with a compound of formula XX or XXI, 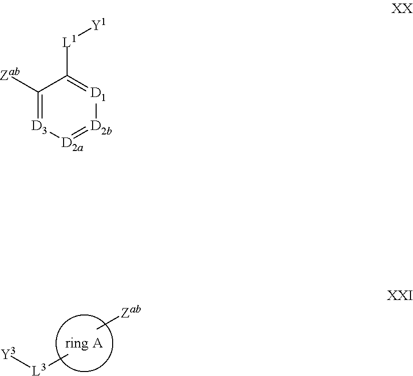
wherein (in all cases) Z^{ab} represents a suitable leaving group, and ring A, D₁, D₂ₐ, D_{2b}, D₃, L¹, L³ and Y³ are as defined in claim 1;
(xvi) for compounds of formula I in which L¹ or, if present, L^{a} represents C₁₋₆ alkylene, and Y¹ and, if present, Y^{1a} preferably represent —C(O)OR^{9b} in which R^{9b} is other than hydrogen, reaction of a compound of formula XXII 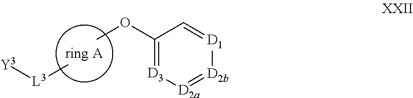
wherein ring A, D₁, D₂ₐ, D_{2b}, D₃, L³ and Y³ are as defined in claim 1, with a compound of formula XXIII,
Z^{aa}-L^{aa}-Y^{aa} XXIII
wherein L^{aa} represents C₁₋₆ alkylene, Y^{aa} represents Y¹ (or Y^{1a}) as defined in claim 1 and Z^{aa} represents a leaving group;
(xvii) for compounds of formula I in which L¹ represents —CH═CH—, reaction of a compound of formula XXIV, 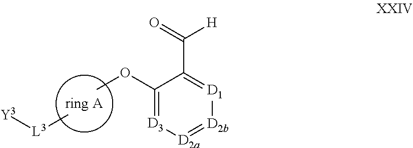
wherein ring A, D₁, D₂ₐ, D_{2b}, D₃, L³ and Y³ are as defined in claim 1, with a compound of formula XXV,
(EtO)₂P(O)CH₂—Y¹ XXV
or the like, or a compound of formula XXVI,
(Ph)₃P═CH—Y¹ XXVI
wherein (in both cases), Y¹ is as defined in claim 1;
(xviii) for compounds of formula I in which L² and/or L³ represent —(CH₂)ₚ—C(O)A¹⁷- in which A¹⁷ represents —N(R^{w})— or —N(R^{w})SO₂—, reaction of a corresponding compound of formula XXVII, 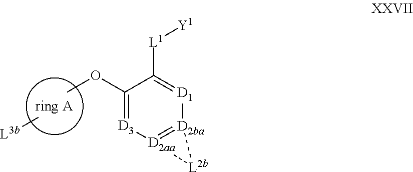
or a protected derivative thereof wherein one of D₂ₐₐ and D_{2ba} represents D₂ and the other represents —C(-L^{2b})═, L^{2b} represents —(CH₂)ₚ—C(O)OH or -L²-Y², L^{3b} represents —(CH₂)ₚ—C(O)OH or -L³-Y³, provided that at least one of L^{2b} and L^{3b} represents —(CH₂)ₚ—C(O)OH, and ring A, D₁, D₂, D₃, L¹ and Y¹ are as defined in claim 1, with a compound of formula XXVIII,
H(R^{w})N-Q^{a}-Y^{a} XXVIII
wherein Q^{a} represents a direct bond or —S(O)₂—, and R^{w} and Y^{a} are as defined in claim 1;
(xix) for compounds of formula I in which L¹-Y¹ represents —C(O)N(H)SO₂R^{9a}, reaction of a corresponding compound of formula XXIX, 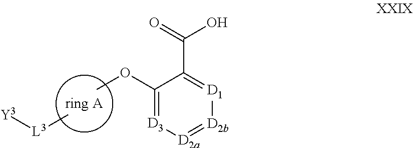
wherein A, D₁, D₂ₐ, D_{2b}, D₃, L³ and Y³ are as defined in claim 1, with a compound of formula XXX,
H₂N—SO₂R^{9a} XXX
wherein R^{9a} is as defined in claim 1, or conversion of the carboxylic acid group of the compound of formula XXIX to the corresponding acyl chloride, followed by reaction of that acyl chloride with a compound of formula XXX;
(xx) for compounds of formula I in which L¹-Y¹ represents —C(O)N(H)SO₂R^{9a}, reaction of a corresponding compound of formula XXXI, 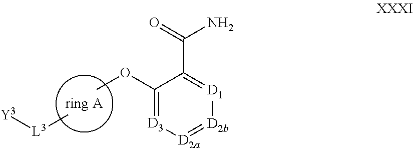
wherein A, D₁, D₂ₐ, D_{2b}, D₃, L³ and Y³ are as defined in claim 1, with a compound of formula XXXII,
Cl—SO₂R^{9a} XXXII
wherein R^{9a} is as defined in claim 1;
(xxi) for compounds of formula I in which L² or L³ represent —N(H)—CH₂—, reductive amination of a compound of formula III as defined above, with a compound of formula XXXIII,
Y^{a}—C(O)H XXXIII
wherein Y^{a} is as defined in process (ii) above.

32. A process for the preparation of a pharmaceutical formulation as defined in claim 21, which process comprises bringing into association a compound of formula I, as defined in claim 1 but without the provisos, or a pharmaceutically acceptable salt thereof with a pharmaceutically-acceptable adjuvant, diluent or carrier.

33. A process for the preparation of a combination product as defined in claim 28, which process comprises bringing into association a compound of formula I, as defined in claim 1 but without the provisos, or a pharmaceutically acceptable salt thereof with the other therapeutic agent that is useful in the treatment of a respiratory disorder and/or inflammation, and at least one pharmaceutically-acceptable adjuvant, diluent or carrier.

34. A use as claimed in claim 23, wherein the disease is a respiratory disease, inflammation and/or has an inflammatory component.
